(19) United States
(12) Patent Application Publication
(10) Pub. No.: US 2010/0004284 A1
(43) Pub. Date: Jan. 7, 2010

(54) Novel Heterocyclic Compounds as Positive Allosteric Modulators of Metabotropic Glutamate Receptors

(75) Inventors: Marco Farina, Milan (IT); Stefania Gagliardi, Milan (IT); Emmanuel Le Poul, Geneva (CH); Giovanni Palombi, Milan (IT); Jean-Philippe Rocher, Plan-les-Ouates (CH)

Correspondence Address: EDWARDS ANGELL PALMER & DODGE LLP, P.O. BOX 55874, BOSTON, MA, 02205, US

(73) Assignee: ADDEX PHARMA SA, GENEVA (CH)

(21) Appl. No.: 11/920,597

(22) PCT Filed: May 17, 2006
(86) PCT No.: PCT/IB2006/001882

§ 371 Date: Sep. 1, 2009

(30) Foreign Application Priority Data
May 18, 2005 (GB) 0510139.9

(51) Int. Cl. A61K 31/4545; C07D 401/04; C07D 211/84; A61K 31/454; A61P 25/18

(52) U.S. Cl. 514/318; 546/209; 546/194; 514/326

(57) ABSTRACT

The present invention relates to new compounds which are Heterocyclic derivatives of formula (I) wherein A, B, P, X, Y, Q, W, R₁ and R₂ are defined in the description. Invention compounds are useful for treating central or peripheral nervous system disorders and other disorders which are affected by the neuromodulatory effect of mGluR5 positive allosteric modulators such as cognitive decline and also to treat both positive and negative symptoms in schizophrenia.

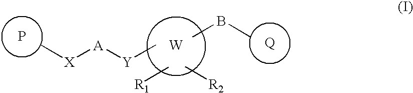

### FIELD OF THE INVENTION

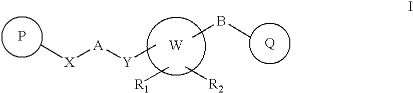

The present invention provides new compounds of formula I as positive allosteric modulators of metabotropic receptors—subtype 5 (“mGluR5”) which are useful for the treatment or prevention of central nervous system disorders such as for example: cognitive decline, both positive and negative symptoms in schizophrenia as well as other central or peripheral nervous system disorders in which the mGluR5 subtype of glutamate metabotropic receptor is involved. The invention is also directed to pharmaceutical compounds and compositions in the prevention or treatment of such diseases in which mGluR5 is involved.

### BACKGROUND OF THE INVENTION

Glutamate, the major amino-acid transmitter in the mammalian central nervous system (CNS), mediates excitatory synaptic neurotransmission through the activation of ionotropic glutamate receptors receptor-channels (iGluRs, namely NMDA, AMPA and kainate) and metabotropic glutamate receptors (mGluRs). iGluRs are responsible for fast excitatory transmission (Nakanishi S et al., (1998) Brain Res. Rev., 26:230-235) while mGluRs have a more modulatory role that contributes to the fine-tuning of synaptic efficacy. Glutamate performs numerous physiological functions such as long-term potentiation (LTP), a process believed to underlie learning and memory but also cardiovascular regulation, sensory perception, and the development of synaptic plasticity. In addition, glutamate plays an important role in the patho-physiology of different neurological and psychiatric diseases, especially when an imbalance in glutamatergic neurotransmission occurs.

The mGluRs are seven-transmembrane G protein-coupled receptors. The eight members of the family are classified into three groups (Groups I, II & III) according to their sequence homology and pharmacological properties (Schoepp D D et al. (1999) Neuropharmacology, 38:1431-1476). Activation of mGluRs lead to a large variety of intracellular responses and activation of different transductional cascades. Among mGluR members, the mGluR5 subtype is of high interest for counterbalancing the deficit or excesses of neurotransmission in neuropsychiatric diseases. mGluR5 belongs to Group I and its activation initiates cellular responses through G-protein mediated mechanisms. mGluR5 is coupled to phospholipase C and stimulates phosphoinositide hydrolysis and intracellular calcium mobilization.

mGluR5 proteins have been demonstrated to be localized in post-synaptic elements adjacent to the post-synaptic density (Lujan R et al. (1996) Eur. J. Neurosci., 8:1488-500; Lujan R et al. (1997) J. Chem. Neuroanat., 13:219-41) and are rarely detected in the pre-synaptic elements (Romano C et al. (1995) J. Comp. Neurol., 355:455-69). mGluR5 receptors can therefore modify the post-synaptic responses to neurotransmitter or regulate neurotransmitter release.

In the CNS, mGluR5 receptors are abundant mainly throughout the cortex, hippocampus, caudate-putamen and nucleus accumbens. As these brain areas have been shown to be involved in emotion, motivational processes and in numerous aspects of cognitive function, mGluR5 modulators are predicted to be of therapeutic interest.

A variety of potential clinical indications have been suggested to be targets for the development of subtype selective mGluR modulators. These include epilepsy, neuropathic and inflammatory pain, numerous psychiatric disorders (e.g. anxiety and schizophrenia), movement disorders (e.g. Parkinson disease), neuroprotection (stroke and head injury), migraine and addiction/drug dependency (for reviews, see Brauner-Osborne H et al. (2000) J. Med. Chem., 43:2609-45; Bordi F and Ugolini A. (1999) Prog. Neurobiol., 59:55-79; Spooren W et al. (2003) Behav. Pharmacol., 14:257-77).

The hypothesis of hypofunction of the glutamatergic system as reflected by NMDA receptor hypofunction as a putative cause of schizophrenia has received increasing support over the past few years (Goff D C and Coyle J T (2001) Am. J. Psychiatry, 158:1367-1377; Carlsson A et al. (2001) Annu. Rev. Pharmacol. Toxicol., 41:237-260 for a review). Evidence implicating dysfunction of glutamatergic neurotransmission is supported by the finding that antagonists of the NMDA subtype of glutamate receptor can reproduce the full range of symptoms as well as the physiologic manifestation of schizophrenia such as hypofrontality, impaired prepulse inhibition and enhanced subcortical dopamine release. In addition, clinical studies have suggested that mGluR5 allele frequency is associated with schizophrenia among certain cohorts (Devon R S et al. (2001) Mol. Psychiatry., 6:311-4) and that an increase in mGluR5 message has been found in cortical pyramidal cells layers of schizophrenic brain (Ohnuma T et al. (1998) Brain Res. Mol. Brain. Res., 56:207-17).

The involvement of mGluR5 in neurological and psychiatric disorders is supported by evidence showing that in vivo activation of group I mGluR5 induces a potentiation of NMDA receptor function in a variety of brain regions mainly through the activation of mGluR5 receptors (Mannaioni G et al. (2001) Neurosci., 21:5925-34; Awad H et al. (2000) J. Neurosci., 20:7871-7879; Pisani A et al. (2001) Neuroscience, 106:579-87; Benquet P et al (2002) J. Neurosci., 22:9679-86).

The role of glutamate in memory processes also has been firmly established during the past decade (Martin S J et al. (2000) Annu. Rev. Neurosci., 23:649-711; Baudry M and Lynch G. (2001) Neurobiol. Learn. Mem., 76:284-297). The use of mGluR5 null mutant mice have strongly supported a role of mGluR5 in learning and memory. These mice show a selective loss in two tasks of spatial learning and memory, and reduced CA1 LTP (Lu et al. (1997) J. Neurosci., 17:5196-5205; Schulz B et al. (2001) Neuropharmacology, 41:1-7; Jia Z et al. (2001) Physiol. Behav., 73:793-802; Rodrigues et al. (2002) J. Neurosci., 22:5219-5229).

The finding that mGluR5 is responsible for the potentiation of NMDA receptor mediated currents raises the possibility that agonists of this receptor could be useful as cognitive-enhancing agents, but also as novel antipsychotic agents that act by selectively enhancing NMDA receptor function.

The activation of NMDARs could potentiate hypofunctional NMDARs in neuronal circuitry relevant to schizophrenia Recent in vivo data strongly suggest that mGluR5 activation may be a novel and efficacious approach to treat cognitive decline and both positive and negative symptoms in schizophrenia (Kinney G G et al. (2003) J. Pharmacol. Exp. Ther., 306(1):116-123).

mGluR5 receptor is therefore being considered as a potential drug target for treatment of psychiatric and neurological disorders including treatable diseases in this connection are anxiety disorders, attentional disorders, eating disorders, mood disorders, psychotic disorders, cognitive disorders, personality disorders and substance-related disorders.

Most of the current modulators of mGluR5 function have been developed as structural analogues of glutamate, quisqualate or phenylglycine (Schoepp D D et al. (1999) Neuropharmacology, 38:1431-1476) and it has been very challenging to develop in vivo active and selective mGluR5 modulators acting at the glutamate binding site. A new avenue for developing selective modulators is to identify molecules that act through allosteric mechanisms, modulating the receptor by binding to site different from the highly conserved orthosteric binding site.

Positive allosteric modulators of mGluRs have emerged recently as novel pharmacological entities offering this attractive alternative. This type of molecule has been discovered for mGluR1, mGluR2, mGluR4, and mGluR5 (Knoflach F et al. (2001) Proc. Natl. Acad. Sci. USA., 98:13402-13407; O'Brien J A et al. (2003) Mol. Pharmacol., 64:731-40; Johnson K et al. (2002) Neuropharmacology, 43:291; Johnson M P et al. (2003) J. Med. Chem., 46:3189-92; Marino M J et al. (2003) Proc. Natl. Acad. Sci. USA., 100(23):13668-73; for a review see Mutel V (2002) Expert Opin. Ther. Patents, 12:1-8; Kew J N (2004) Pharmacol. Ther., 104(3):233-44; Johnson M P et al. (2004) Biochem. Soc. Trans., 32:881-7). DFB and related molecules were described as in vitro mGluR5 positive allosteric modulators but with low potency (O'Brien J A et al. (2003) Mol. Pharmacol., 64:731-40). Benzamide derivatives have been patented (WO 2004/087048; O'Brien J A (2004) J. Pharmacol. Exp. Ther., 309:568-77) and recently aminopyrazole derivatives have been disclosed as mGluR5 positive allosteric modulators (Lindsley et al. (2004) J. Med. Chem., 47:5825-8; WO 2005/087048). Among aminopyrazole derivatives, CDPPB has shown in vivo activity antipsychotic-like effects in rat behavioral models (Kinney G G et al. (2005) J. Pharmacol. Exp. Ther., 313:199-206). This report is consistent with the hypothesis that allosteric potentiation of mGluR5 may provide a novel approach for development of antipsychotic agents. Recently a novel series of positive allosteric modulators of mGluR5 receptors has been disclosed (WO 2005/044797). Aryloxadiazole derivatives have been patented (WO 04/014370); these compounds are negative allosteric modulators of mGluR5 receptors. Several classes of aryl and heteroaryloxadiazole compounds have been disclosed: WO 01/54507, WO 03/056823, WO 02/72570, GB 1164572, FR 6671). Benzoyl triazoles with affinity for serotonergic receptors have been published (Caliendo G et al. (1999) Eur. J. Med. Chem., 34, 9, 719-727; Caliendo G. et al. (2002) Eur. J. Pharm. Sci., 16, 1-2, 15-28). U.S. Pat. No. 3,509,153 to Hayao et al. discloses hypotensive 2-(substituted-propyl)tetrazole salts.

None of the specifically disclosed compounds are structurally related to the compounds of the present invention.

The present invention relates to a method of treating or preventing a condition in a mammal, including a human, the treatment or prevention of which is affected or facilitated by the neuromodulatory effect of mGluR5 positive allosteric modulators.

### FIGURE

FIG. 1 shows the effect of 10 μM of example #1 of the present invention on primary cortical mGluR5-expressing cell cultures in the absence or in the presence of 300 nM glutamate.

### DETAILED DESCRIPTION OF THE INVENTION

According to the present invention, there are provided new compounds of the general formula I

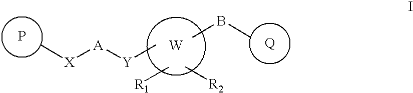

Or pharmaceutically acceptable salts, hydrates or solvates of such compounds

### Wherein

W represents (C₅-C₇)cycloalkyl, (C₅-C₇)heterocycloalkyl or (C₅-C₇)heterocycloalkenyl ring;
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P and Q are each independently selected and denote a cycloalkyl, a heterocycloalkyl, an aryl or heteroaryl group of formula

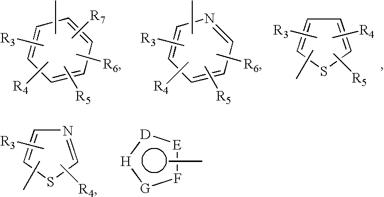

R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR %, —S(═O)R₈, —S(═O)₂R₈, —S(═O)₂NR₈R₉, —C(═O) %, —COOR₈, —C(═O)NR₈R₉, —C(═NR₈)R₉, or C(—NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(—S)—, —O—, —N═, —N(R₃)— or —S—;
A is azo —N═N—, ethyl, ethenyl, ethynyl, —NR₈C(═O)—, —NR₈S(═O)₂—, —C(═O)NR₈—, —S—, —S(═O)—, —S(═O)₂—, —S(═O)₂NR₈—, —C(═O)—O—, —O—C(═O)—, —C(═NR₈)NR₉—, —C(═NOR₈)NR₉—, —NR₈C(═NOR₉)—, ═N—O—, —O—N═CH— or a group aryl or heteroaryl of formula

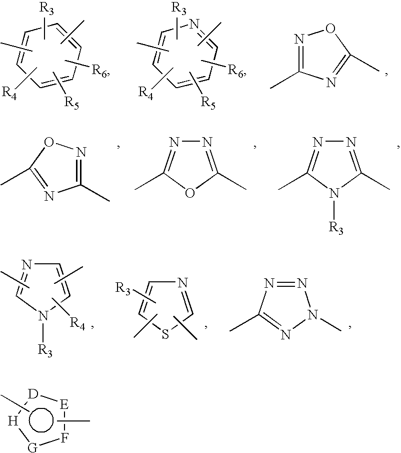

R₃, R₄, R₅ and R₆ independently are as defined above;
D, E, F, G and H in A independently represent a carbon group, oxygen, nitrogen, sulphur or a double bond;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R₈ and R₉, independently are as defined above;
X and Y are each independently selected from a bond, —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)allyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁—C(═O)—(C₀-C₆)alkyl-, —(C₀-C₆)alkyl-NR¹¹C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(—S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
   X and Y together cannot be a bond;
   R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆-alkyl), —O—(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
   Any N may be an N-oxide.

The present invention includes both possible stereoisomers and includes not only racemic compounds but the individual enantiomers as well.

For the avoidance of doubt it is to be understood that in this specification “(C₁-C₆)” means a carbon group having 1, 2, 3, 4, 5 or 6 carbon atoms. “(C₀-C₆)” means a carbon group having 0, 1, 2, 3, 4, 5 or 6 carbon atoms. In this specification “C” means a carbon atom.

In the above definition, the term “(C₁-C₆)alkyl” includes group such as methyl, ethyl, propyl, isopropyl, butyl, isobutyl, sec-butyl, tert-butyl, pentyl, isopentyl, neopentyl, tert-pentyl, hexyl or the like.

“(C₂-C₆)alkenyl” includes group such as ethenyl, 1-propenyl, allyl, isopropenyl, 1-butenyl, 3-butenyl, 4-pentenyl and the like.

“(C₂-C₆)alkynyl” includes group such as ethynyl, propynyl, butynyl, pentynyl and the like.

“Halogen” includes atoms such as fluorine, chlorine, bromine and iodine.

“Cycloalkyl” refers to an optionally substituted carbocycle containing no heteroatoms, includes mono-, bi-, and tricyclic saturated carbocycles, as well as fused ring systems. Such fused ring systems can include on ring that is partially or fully unsaturated such as a benzene ring to form fused ring systems such as benzo fused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, fluorenyl, 1,2,3,4-tetrahydronaphthalene and the like.

“Heterocycloalkyl” refers to an optionally substituted carbocycle containing at least one heteroatom selected independently from O, N, S. It includes mono-, bi-, and tricyclic saturated carbocycles, as well as fused ring systems. Such fused ring systems can include one ring that is partially or fully unsaturated such as a benzene ring to form fused ring systems such as benzo fused carbocycles. Examples of heterocycloalkyl include piperidine, piperazine, morpholine, tetrahydrothiophene, indoline, isoquinoline and the like.

“Aryl” includes (C₆-C₁₀)aryl group such as phenyl, 1-naphtyl, 2-naphtyl and the like.

“Arylalkyl” includes (C₆-C₁₀)aryl-(C₁-C₃)alkyl group such as benzyl group, 1-phenylethyl group, 2-phenylethyl group, 1-phenylpropyl group, 2-phenylpropyl group, 3-phenylpropyl group, 1-naphtylmethyl group, 2-naphtylmethyl group or the like.

“Heteroaryl” includes 5-10 membered heterocyclic group containing 1 to 4 heteroatoms selected from oxygen, nitrogen or sulphur to form a ring such as furyl (furan ring), benzofuranyl (benzofuran ring), thienyl (thiophene ring), benzothiophenyl (benzothiophene ring), pyrrolyl (pyrrole ring), imidazolyl (imidazole ring), pyrazolyl (pyrazole ring), thiazolyl (thiazole ring), isothiazolyl (isothiazole ring), triazolyl (triazole ring), tetrazolyl (tetrazole ring), pyridil (pyridine ring), pyrazynyl (pyrazine ring), pyrimidinyl (pyrimidine ring), pyridazinyl (pyridazine ring), indolyl (indole ring), isoindolyl (isoindole ring), benzoimidazolyl (benzimidazole ring), purinyl group (purine ring), quinolyl (quinoline ring), phtalazinyl (phtalazine ring), naphtyridinyl (naphtyridine ring), quinoxalinyl (quinoxaline ring), cinnolyl (cinnoline ring), pteridinyl (pteridine ring), oxazolyl (oxazole ring), isoxazolyl (isoxazole ring), benzoxazolyl (benzoxazole ring), benzothiazolyl)-(benzothiaziole ring), furazanyl (furazan ring) and the like.

“Heteroarylalkyl” includes heteroaryl-(C₁-C₃-alkyl) group, wherein examples of heteroaryl are the same as those illustrated in the above definition, such as 2-furylmethyl group, 3-furylmethyl group, 2-thienylmethyl group, 3-thienylmethyl group, 1-imidazolylmethyl group, 2-imidazolylmethyl group, 2-thiazolylmethyl group, 2-pyridylmethyl group, 3-pyridylmethyl group, 1-quinolylmethyl group or the like.

“Solvate” refers to a complex of variable stochiometry formed by a solute (e.g. a compound of formula I) and a solvent. The solvent is a pharmaceutically acceptable solvent as water preferably; such solvent may not interfere with the biological activity of the solute.

“Optionally” means that the subsequently described event(s) may or may not occur, and includes both event(s), which occur, and events that do not occur.

The term “substituted” refers to substitution with the named substituent or substituents, multiple degrees of substitution being allowed unless otherwise stated.

Preferred compounds of the present invention are compounds of formula I-A depicted below

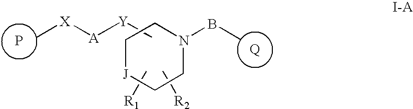

or pharmaceutically acceptable salts, hydrates or solvates of such compounds.

### Wherein

R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P and Q are each independently selected and denote a cycloalkyl, a heterocycloalkyl, an aryl or heteroaryl group of formula

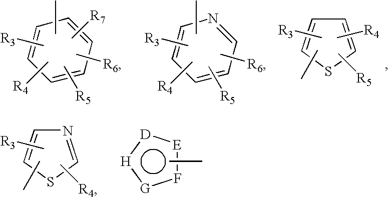

R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(—NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
A is azo —N═N—, ethyl, ethenyl, ethynyl, —NR₈C(═O)—, —NR₈S(═O)₂—, —C(═O)NR₈—, —S—, —S(═O)—, —S(═O)₂—, —S(═ON—, —C(═O)—O—, —O—C(═O)—, —C(═NR₈)NR₉—, —C(═NOR₈)NR₉—, —NR₈C(—NOR₉)—, ═N—O—, —O—N═CH— or a group aryl or heteroaryl of formula

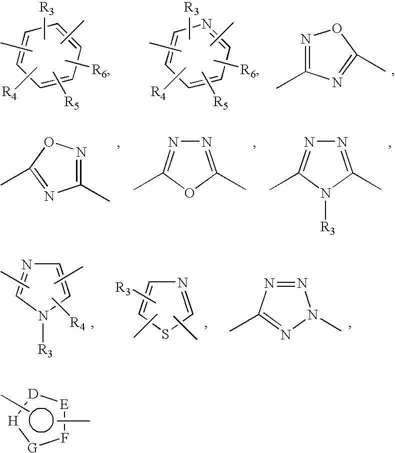

R₃, R₄, R₅ and R₆ independently are as defined above;
D, E, F, G and H in A independently represent a carbon group, oxygen, nitrogen, sulphur or a double bond;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R₈ and R₉, independently are as defined above;
X and Y are each independently selected from a bond, —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)arylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁—C(═O)—(C₀-C₆)alkyl-, —(C₀-C₆)alkyl-NR¹¹C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, (C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
   X and Y together cannot be a bond;
   R₁, and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
   R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

Particularly preferred compounds of the present invention are compounds of formula I-B

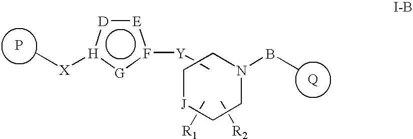

or pharmaceutically acceptable salts, hydrates or solvates of such compounds.

### Wherein

R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P and Q are each independently selected and denote a cycloalkyl, a heterocycloalkyl, an aryl or heteroaryl group of formula

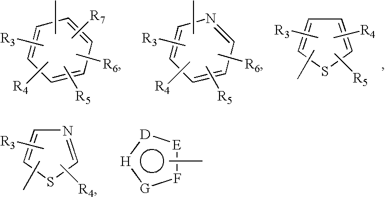

R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(—NOR₈)NR₉—(C₀-C₂)alkyl-;
   R₈ and R₉, independently are as defined above;
X and Y are each independently selected from a bond, —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)allyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)allyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR₁₁C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)allyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
   X and Y together cannot be a bond;
   R₁, and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—; R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alknyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

Further preferred compounds of the present invention are compounds of formula I-C

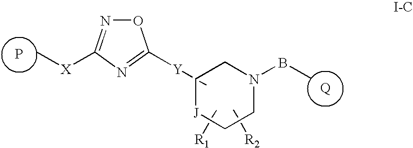

or pharmaceutically acceptable salts, hydrates or solvates of such compounds.

### Wherein

R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P and Q are each independently selected and denote a cycloalkyl, a heterocycloalkyl, an aryl or heteroaryl group of formula

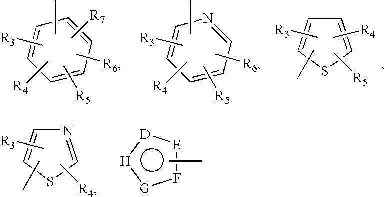

R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₉, —S(═O)R₈, —S(═O)₂R₈, —S(═O)₂NR₉R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
   R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
   D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(—NR₈)—(C₀-C₂)alkyl-, —C(═NOR₈)—(C₀-C₂)alkyl- or —C(═NOR₉)NR₉—(C₀-C₂)alkyl-;
   R₈ and R₉, independently are as defined above;
X and Y are each independently selected from a bond, —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(—O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)allyl-NR₁₁C(═O)—(C₀-C₆)alkyl-, —(C₀-C₆)alkyl-NR₁₁C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
   X and Y together cannot be a bond;
   R₁, and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
   R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

In another aspect, the compound of this invention is represented by formula (I-D)

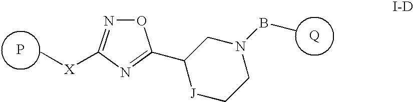

or pharmaceutically acceptable salts, hydrates or solvates of such compounds.

### Wherein

P and Q are each independently selected and denote a cycloalkyl, a heterocycloalkyl, an aryl or heteroaryl group of formula

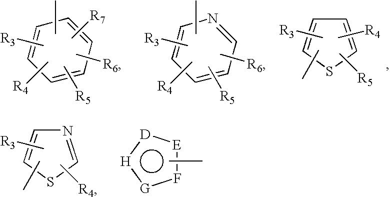

R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R₈ and R₉, independently are as defined above;
X represents —NR₁₁C(═O)O—, an optionally substituted —(C₁-C₆)alkyl-, —(C₂-C₆)alkyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)allyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR₁₁C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁, —(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁, —(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl; R₁₁ and R₁₂ each independently is hydrogen, C₁-C₆-allyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
   R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

In further aspect, the compound of this invention is represented by formula (I-D) or pharmaceutically acceptable salts, hydrates or solvates of such compounds.

### Wherein

X represents an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl- or —(C₀-C₆)alkyl-NR₁₁—(C₄-C₁₀)alkylcycloalkyl-;
   R₁₁ is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
   Any N may be an N-oxide;

The present invention includes both possible stereoisomers and includes not only racemic compounds but the individual enantiomers as well.

Another aspect of the invention are compounds of the formula II-A

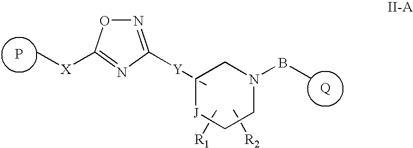

or pharmaceutically acceptable salts, hydrates or solvates of such compounds.

### Wherein

R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P and Q are each independently selected and denote a cycloalkyl, a heterocycloalkyl, an aryl or heteroaryl group of formula

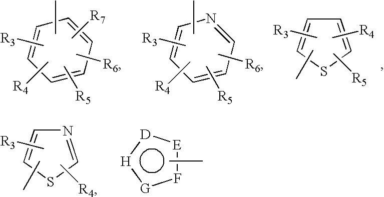

R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₉, —S(═O)R₈, —S(═O)₂R₈, —S(═O)₂NR₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
   D, E, F, G and H in P and Q represent independently C(R₃)═, —C(R₃)═C(R₄)—, —C(═O)—, —C(—S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R₈ and R₉, independently are as defined above;
X and Y are each independently selected from a bond, —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-2-(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR₁₁C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(—O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₀C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
   X and Y together cannot be a bond;
   R₁, and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond; —C(R₁₃)(R₁₄), —O—, —N(R₁₃)— or —S—;
   R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

An embodiment of the present invention includes compounds of the formula II-B

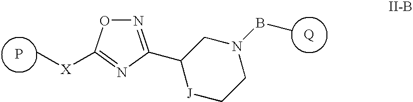

or pharmaceutically acceptable salts, hydrates or solvates of such compounds.

### Wherein

P and Q are each independently selected and denote a cycloalkyl, a heterocycloalkyl, an aryl or heteroaryl group of formula

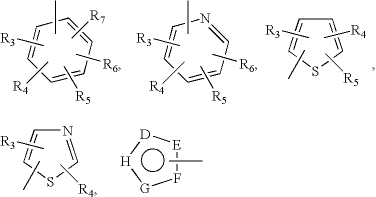

R₃, R₄, R₅, R₆, and R₇ independently are hydrogen, halogen, —CN, NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(—NR₈)NR₉—S(═O)—(C₀-C₂)alkyl-, —S(═O)—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R₈ and R₉, independently are as defined above;
X represents —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)₄alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)allyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR₁₁C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
   R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
   R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

An embodiment of the present invention includes compounds of the formula II-B

Wherein
X represents an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl- or —(C₀-C₆)alkyl-NR₁₁—(C₄-C₁₀)alkylcycloalkyl-;
   R₁₁ is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
   Any N may be an N-oxide;

The present invention includes both possible stereoisomers and includes not only racemic compounds but the individual enantiomers as well.

An embodiment of the present invention includes compounds of the formula III-A

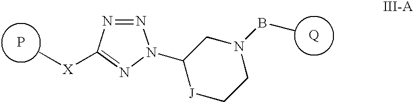

or pharmaceutically acceptable salts, hydrates or solvates of such compounds.

### Wherein

P and Q are each independently selected and denote a cycloalkyl, a heterocycloalkyl, an aryl or heteroaryl group of formula

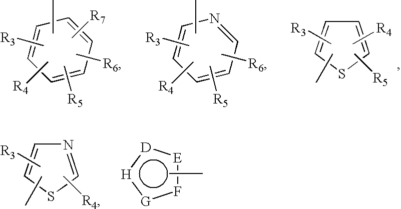

R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₉, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
   D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R₈ and R₉, independently are as defined above;
X represents an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl- or —(C₀-C₆)alkyl-NR₁₁—(C₄-C₁₀)alkylcycloalkyl-;
   R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—; R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

An embodiment of the present invention includes compounds of the formula IV-A

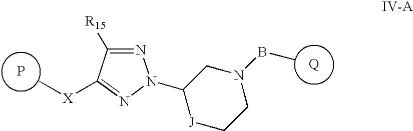

or pharmaceutically acceptable salts, hydrates or solvates of such compounds.

### Wherein

P and Q are each independently selected and denote a cycloalkyl, a heterocycloalkyl, an aryl or heteroaryl group of formula

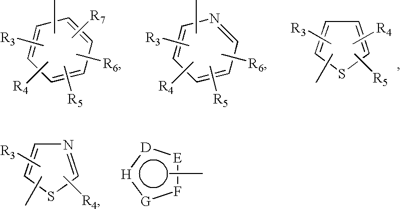

R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(—NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)allyl-, —C(═NOR₈)—(C₀-C₂)alkyl- or —C(═NOR₉)NR₉—(C₀-C₂)alkyl-;
   R₈ and R₉, independently are as defined above;
X represents an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C_{O-}C₆)alkyl-O—(C₂-C₆)alkynyl-, —(C₀-C₆)allyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, 7(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl- or —(C₀-C₆)alkyl-NR₁₁—(C₄-C₁₀)alkylcycloalkyl-;
   R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
   R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
R₁₅ is hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl, —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

Specifically preferred compounds are:
{(S)-3-[3-(4-Fluoro-benzyl)-[1,2,4]oxadiazol-5-yl]-piperidin-1-yl}-(4-fluoro-phenyl)-methanone,
(3,4-Difluoro-phenyl)-{(S)-3-[3-(4-fluoro-benzyl)-[1,2,4]oxadiazol-5-yl]-piperidin-1-yl}-methanone
(3,4-Difluoro-phenyl)-{(S)-3-[5-(4-fluoro-benzyl)-[1,2,4]oxadiazol-3-yl]-piperidin-1-yl}-methanone
{(S)-3-[5-(4-Fluoro-benzyl)-[1,2,4]oxadiazol-3-yl]-piperidin-1-yl}-(4-fluoro-phenyl)-methanone
(4-Fluoro-phenyl)-{(S)-3-[5-((S)-1-phenyl-ethyl)-[1,2,4]oxadiazol-3-yl]-piperidin-1-yl}-methanone
(4-Fluoro-phenyl)-{(S)-3-[5-((R)-1-phenyl-ethyl)-[1,2,4]oxadiazol-3-yl]-piperidin-1-yl}-methanone
[(S)-3-(5-Benzyl-[1,2,4]oxadiazol-3-yl)-piperidin-1-yl]-(4-fluoro-phenyl)-methanone
(4-Fluoro-phenyl)-{(S)-3-[5-((S)-hydroxy-phenyl-methyl)-[1,2,4]oxadiazol-3-yl]-piperidin-1-yl}-methanone
(4-Fluoro-phenyl)-{(S)-3-[5-((R)-hydroxy-phenyl-methyl)-[1,2,4]oxadiazol-3-yl]-piperidin-1-yl}-methanone
(4-Fluoro-phenyl)-[(S)-3-(5-phenethyl-[1,2,4]oxadiazol-3-yl)-piperidin-1-yl]-methanone
{3-[(S)-1-(4-Fluoro-benzoyl)-piperidin-3-yl]-[1,2,4]oxadiazol-5-yl}-phenyl-methanone
(4-Fluoro-phenyl)-[(S)-3-(5-phenylamino-[1,2,4]oxadiazol-3-yl)-piperidin-1-yl]-methanone
{(S)-3-[5-(4-Fluoro-benzylamino)-[1,2,4]oxadiazol-3-yl]-piperidin-1-yl}-(4-fluoro-phenyl)-methanone
[(S)-3-(5-Benzyl-tetrazol-2-yl)-piperidin-1-yl]-(4-fluoro-phenyl)-methanone
{3-[3-(4-Fluoro-phenoxy)-[1,2,4]oxadiazol-5-yl]-piperidin-1-yl}-(4-fluoro-phenyl)-methanone
(4-Fluoro-phenyl)-[3-(3-phenoxy-[1,2,4]oxadiazol-5-yl)-piperidin-1-yl]-methanone
(6-Fluoro-pyridin-3-yl)-[(S)-3-(3-phenoxy-[1,2,4]oxadiazol-5-yl)-piperidin-1-yl]-methanone
{(S)-3-[3-(2-Fluoro-phenoxy)-[1,2,4]oxadiazol-5-yl]-piperidin-1-yl}-(6-fluoro-pyridin-3-yl)-methanone
{(S)-3-[3-(3-Fluoro-phenoxy)-[1,2,4]oxadiazol-5-yl]-piperidin-1-yl}-(6-fluoro-pyridin-3-yl)-methanone
(4-Fluoro-phenyl)-[(S)-3-(3-phenylsulfanyl-[1,2,4]oxadiazol-5-yl)-piperidin-1-yl]-methanone
{3-[3-(3-Fluoro-phenoxy)-[1,2,4]oxadiazol-5-yl]-piperidin-1-yl}-(4-fluoro-phenyl)-methanone
{3-[3-(3-Fluoro-phenoxy)-[1,2,4]oxadiazol-5-yl]-piperidin-1-yl}-(4-fluoro-phenyl)-methanone
(4-Methylphenyl)-[(S)-3-(3-phenoxy-[1,2,4]oxadiazol-5-yl)-piperidin-1-yl]-methanone
(2-Methoxy-phenyl)-[(S)-3-(3-phenoxy-[1,2,4]oxadiazol-5-yl)-piperidin-1-yl]-methanone
[(S)-3-(3-Phenoxy-[1,2,4]oxadiazol-5-yl)-piperidin-1-yl]-pyridin-2-yl-methanone
(2-Fluoro-pyridin-4-yl)-[(S)-3-(3-phenoxy-[1,2,4]oxadiazol-5-yl)-piperidin-1-yl]-methanone
(3H-Imidazol-4-yl-[(S)-3-(3-phenoxy-[1,2,4]oxadiazol-5-yl)-piperidin-1-yl]-methanone
(3,5-Difluoro-phenyl)-[(S)-3-(3-phenoxy-[1,2,4]oxadiazol-5-yl)-piperidin-1-yl]-methanone
(5-Methyl-isoxazol-4-yl)-[(S)-3-(3-phenoxy-[1,2,4]oxadiazol-5-yl)-piperidin-1-yl]-methanone
[(S)-3-(3-Phenoxy-[1,2,4]oxadiazol-5-yl)-piperidin-1-yl]-thiazol-5-yl-methanone
[(S)-3-(3-Phenoxy-[1,2,4]oxadiazol-5-yl)-piperidin-1-yl]-phenyl-methanone
(4-Chloro-phenyl)-[(S)-3-(3-phenoxy-[1,2,4]oxadiazol-5-yl)-piperidin-1-yl]-methanone
(4-Methoxy-phenyl)-[(S)-3-(3-phenoxy-[1,2,4]oxadiazol-5-yl)-piperidin-1-yl]-methanone
(3,4-Dichloro-phenyl)-[(S)-3-(3-phenoxy-[1,2,4]oxadiazol-5-yl)-piperidin-1-yl]-methanone
(3-Methoxy-phenyl)-[(S)-3-(3-phenoxy-[1,2,4]oxadiazol-5-yl)-piperidin-1-yl]-methanone
(2-Methyl-phenyl)-[(S)-3-(3-phenoxy-[1,2,4]oxadiazol-5-yl)-piperidin-1-yl]-methanone
(2-Fluoro-phenyl)-[(S)-3-(3-phenoxy-[1,2,4]oxadiazol-5-yl)-piperidin-1-yl]-methanone
(3-Fluoro-phenyl)-[(S)-3-(3-phenoxy-[1,2,4]oxadiazol-5-yl)-piperidin-1-yl]-methanone
[(S)-3-(3-Phenoxy-[1,2,4]oxadiazol-5-yl)-piperidin-1-yl]-pyridin-3-yl-methanone
[(S)-3-(3-Phenoxy-[1,2,4]oxadiazol-5-yl)-piperidin-1-yl]-pyridin-4-yl-methanone
(3,5-Dimethyl-isoxazol-4-yl)-[(S)-3-(3-phenoxy-[1,2,4]oxadiazol-5-yl)-piperidin-1-yl]-methanone
(4-Fluoro-phenyl)-[(S)-3-(3-phenoxy-[1,2,4]oxadiazol-5-yl)-piperidin-1-yl]-methanone
{(S)-3-[3-(3-Fluoro-phenoxy)-[1,2,4]oxadiazol-5-yl]-piperidin-1-yl}-(4-fluoro-phenyl)-methanone
{(S)-3-[3-(3-Fluoro-phenoxy)-[1,2,4]oxadiazol-5-yl]-piperidin-1-yl}-p-tolyl-methanone
{(S)-3-[3-(3-Fluoro-phenoxy)-[1,2,4]oxadiazol-5-yl]-piperidin-1-yl}-(2-methoxy-phenyl)-methanone
{(S)-3-[3-(3-Fluoro-phenoxy)-[1,2,4]oxadiazol-5-yl]-piperidin-1-yl}-(2-fluoro-pyridin-4-yl)-methanone
{(S)-3-[3-(3-Fluoro-phenoxy)-[1,2,4]oxadiazol-5-yl]-piperidin-1-yl}-(3H-imidazol-4-yl)-methanone
(3,5-Difluoro-phenyl)-{(S)-3-[3-(3-fluoro-phenoxy)-[1,2,4]oxadiazol-5-yl]-piperidin-1-yl}-methanone
{(S)-3-[3-(3-Fluoro-phenoxy)-[1,2,4]oxadiazol-5-yl]-piperidin-1-yl}-(5-methyl-isoxazol-4-yl)-methanone
{(S)-3-[3-(3-Fluoro-phenoxy)-[1,2,4]oxadiazol-5-yl]-piperidin-1-yl}-thiazol-5-yl-methanone
{(S)-3-[3-(3-Fluoro-phenoxy)-[1,2,4]oxadiazol-5-yl]-piperidin-1-yl}-(6-fluoro-pyridin-3-yl)-methanone
{(S)-3-[3-(3-Fluoro-phenoxy)-[1,2,4]oxadiazol-5-yl]-piperidin-1-yl}-pyridin-2-yl-methanone
{(S)-3-[3-(3-Fluoro-phenoxy)-[1,2,4]oxadiazol-5-yl]-piperidin-1-yl}-phenyl-methanone
(4-Chloro-phenyl)-{(S)-3-[3-(3-fluoro-phenoxy)-[1,2,4]oxadiazol-5-yl]-piperidin-1-yl}-methanone
{(S)-3-[3-(3-Fluoro-phenoxy)-[1,2,4]oxadiazol-5-yl]-piperidin-1-yl}-(4-methoxy-phenyl)-methanone
(3,4-Dichloro-phenyl)-{(S)-3-[3-(3-fluoro-phenoxy)-[1,2,4]oxadiazol-5-yl]-piperidin-1-yl)-methanone
((S)-3-[3-(3-Fluoro-phenoxy)-[1,2,4]oxadiazol-5-yl]-piperidin-1-yl}-(3-methoxy-phenyl)-methanone
{(S)-3-[3-(3-Fluoro-phenoxy)-[1,2,4]oxadiazol-5-yl]-piperidin-1-yl}-o-tolyl-methanone
{(S)-3-[3-(3-Fluoro-phenoxy)-[1,2,4]oxadiazol-5-yl]-piperidin-1-yl}-(2-fluoro-phenyl)-methanone
{(S)-3-[3-(3-Fluoro-phenoxy)-[1,2,4]oxadiazol-5-yl]-piperidin-1-yl}-(3-fluoro-phenyl)-methanone
{(S)-3-[3-(3-Fluoro-phenoxy)-[1,2,4]oxadiazol-5-yl]-piperidin-1-yl}-pyridin-3-yl-methanone
{(S)-3-[3-(3-Fluoro-phenoxy)-[1,2,4]oxadiazol-5-yl]-piperidin-1-yl}-pyridin-4-yl-methanone
{(S)-3-[3-(3-Fluoro-phenoxy)-[1,2,4]oxadiazol-5-yl]-piperidin-1-yl}-(3,5-dimethyl-isoxazol-4-yl)-methanone.

The present invention relates to the pharmaceutically acceptable acid addition salts of compounds of the formula I or pharmaceutically acceptable carriers or excipients.

The present invention relates to a method of treating or preventing a condition in a mammal, including a human, the treatment or prevention of which is affected or facilitated by the neuromodulatory effect of mGluR5 allosteric modulators and particularly positive allosteric modulators.

The present invention relates to a method useful for treating or preventing various peripheral and central nervous system disorders such as tolerance or dependence, anxiety, depression, psychiatric disease such as psychosis, inflammatory or neuropathic pain, memory impairment, Alzheimer's disease, ischemia, drug abuse and addiction, as defined in the attached claims.

The present invention relates to pharmaceutical compositions which provide from about 0.01 to 1000 mg of the active ingredient per unit dose. The compositions may be administered by any suitable route. For example orally in the form of capsules or tablets, parenterally in the form of solutions for injection, topically in the form of onguents or lotions, ocularly in the form of eye-lotion, rectally in the form of suppositories.

The pharmaceutical formulations of the invention may be prepared by conventional methods in the art; the nature of the pharmaceutical composition employed will depend on the desired route of administration. The total daily dose usually ranges from about 0.05-2000 mg.

### Methods of Synthesis

Compounds of general formula I may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (Green T. W. and Wuts P. G. M. (1991) Protecting Groups in Organic Synthesis, John Wiley et Sons). These groups are removed at a convenient stage of the compound synthesis using methods that are readily apparent to those skilled in the art. The selection of process as well as the reaction conditions and order of their execution shall be consistent with the preparation of compounds of formula I.

The compound of formula I may be represented as a mixture of enantiomers, which may be resolved into the individual pure R- or S-enantiomers. If for instance, a particular enantiomer of the compound of formula I is desired, it may be prepared by asymmetric synthesis, or by derivation with a chiral auxiliary, where the resulting diastereomeric mixture is separated and the auxiliary group cleaved to provided the pure desired enantiomers. Alternatively, where the molecule contains a basic functional group such as amino, or an acidic functional group such as carboxyl, this resolution may be conveniently performed by fractional crystallization from various solvents, of the salts of the compounds of formula I with optical active acid or by other methods known in the literature, e.g. chiral column chromatography. Resolution of the final product, an intermediate or a starting material may be performed by any suitable method known in the art as described by Eliel E. L., Wilen S. H. and Mander L. N. (1984) Stereochemistry of Organic Compounds, Wiley-Interscience.

Many of the heterocyclic compounds of formula I where A is an heteroaromatic group can be prepared using synthetic routes well known in the art (Katrizky A. R. and Rees C. W. (1984) Comprehensive Heterocyclic Chemistry, Pergamon Press).

The product from the reaction can be isolated and purified employing standard techniques, such as extraction, chromatography, crystallization, distillation, and the like.

The compounds of formula I-A wherein W is a 3-substituted piperidine ring may be prepared according to the synthetic sequences illustrated in the Schemes 1-4.

Wherein
P and Q each independently is aryl or heteroaryl as described above
X is CH₂,
B represents —C(═O)—(C₀-C₂)alkyl-; —S(═O)₂—(C₀-C₂)alkyl-.

The starting material amidoxime can be prepared by methods known in the art of organic synthesis as set forth in part by the following synthesis Scheme 1.

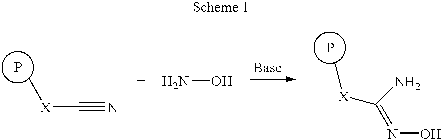

In turn, a nitrile derivative (for example 4-fluoro-benzylnitrile or phenyl cyanate) is reacted with hydroxylamine under neutral or basic conditions such as triethylamine, diisopropyl-ethylamine, sodium carbonate, sodium hydroxide and the like in a suitable solvent (e.g. methyl alcohol, ethyl alcohol). The reaction typically proceeds by allowing the reaction temperature to warm slowly from ambient temperature to a temperature range of 70° C. up to 80° C. inclusive for a time in the range of about 1 hour up to 48 hours inclusive (see for example Lucca, George V. De; Kim, Ui T.; Liang, Jing; Cordova, Beverly; Klabe, Ronald M.; et al; J. Med. Chem.; EN; 41; 13; 1998; 2411-2423, Lila, Christine; Gloanec, Philippe; Cadet, Laurence; Herve, Yolande; Fournier, Jean; et al.; Synth. Commun.; EN; 28; 23; 1998; 4419-4430 and see: Sendzik, Martin; Hui, Hon C.; Tetrahedron Lett.; EN; 44; 2003; 8697-8700 and references therein for reaction under neutral conditions).

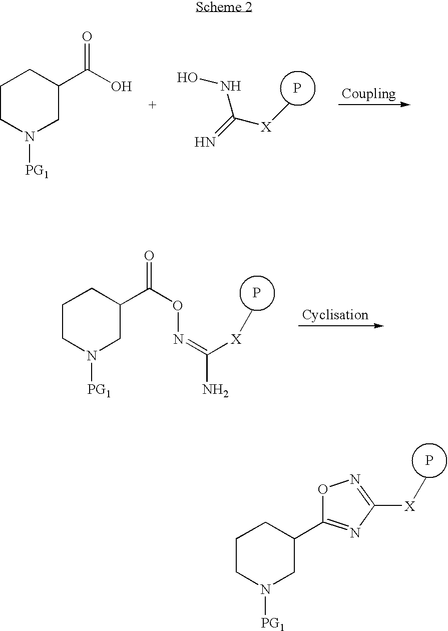

The substituted amidoxime derivative (described in the Scheme 1) may be converted to an acyl-amidoxime derivative using the approach outlined in the Scheme 2. In the Scheme 2, PG₁ is an amino protecting group such as tert-butyloxycarbonyl, benzyloxycarbonyl, ethoxycarbonyl, benzyl and the like. The coupling reaction may be promoted by coupling agents known in the art of organic synthesis such as EDCI (1-(3-dimethylaminopropyl)-3-ethylcarbodiimide), DCC(N,N′-dicyclohexylcarbodiimide), in the presence of a suitable base such as triethylamine, diisopropyl-ethylamine, in a suitable solvent (e.g. tetrahydrofuran, dichloromethane, N,N-dimethylformamide, dioxane). Typically, a co-catalyst such as HOBT (hydroxy-benzotriazole), HOAT (1-hydroxy-7-azabenzotriazole) may also be present in the reaction mixture. The reaction typically proceeds at a temperature in the range of ambient temperature up to 60° C. inclusive for a time in the range of about 2 hours up to 12 hours to produce the intermediate acyl-amidoxime. The cyclisation reaction may be effected thermally in a temperature range of about 80° C. up to about 150° C. for a time in the range of about 2 hours up to 18 hours (see for example Suzuki, Takeshi; Iwaoka, Kiyoshi; Imanishi, Naoki; Nagakura, Yukinori; Miyata, Keiji; et al.; Chem. Pharm. Bull.; EN; 47; 1; 1999; 120-122). The product from the reaction can be isolated and purified employing standard techniques, such as extraction, chromatography, crystallization, distillation, and the like.

The final step may be effected either by a process described in the Scheme 3 or by a process described in the Scheme 4.

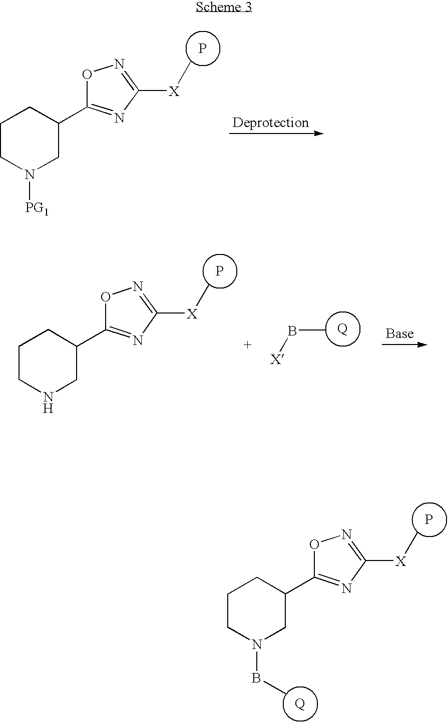

As shown in the Scheme 3, protecting groups PG₁ are removed using standard methods. In the Scheme 3, B is as defined above, X′ is halogen, for example the piperidine derivative is reacted with an aryl or heteroaryl acyl chloride using method that are readily apparent to those skilled in the art. The reaction may be promoted by a base such as triethylamine, diisopropylamine, pyridine in a suitable solvent (e.g. tetrahydrofuran, dichloromethane). The reaction typically proceeds by allowing the reaction temperature to warm slowly from 0° C. up to ambient temperature for a time in the range of about 4 up to 12 hours.

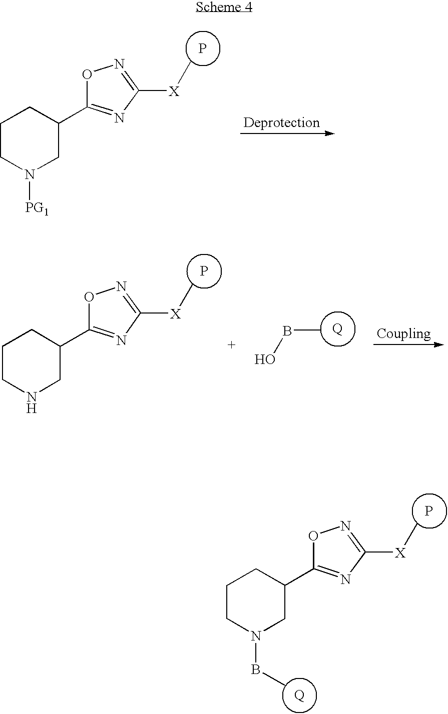

As shown in the Scheme 4, protecting groups PG₁ are removed using standard methods. The coupling reaction may be promoted by coupling agents known in the art of organic synthesis such as EDCI (1-(3-dimethylaminopropyl)-3-ethylcarbodiimide), DCC(N,N′-dicyclohexyl-carbodiimide) or by polymer-supported coupling agents such as polymer-supported carbodiimide (PS-DCC, ex Argonaut Technologies), in the presence of a suitable base such as triethylamine, diisopropyl-ethylamine, in a suitable solvent (e.g. tetrahydrofuran, dichloromethane, N,N-dimethylformamide, dioxane). Typically, a co-catalyst such as HOBT (1-hydroxy-benzotriazole), HOAT (1-hydroxy-7-azabenzotriazole) and the like may also be present in the reaction mixture. The reaction typically proceeds at ambient temperature for a time in the range of about 2 hours up to 12 hours.

The compounds of formula II-B wherein W is a 3-substituted piperidine ring may be prepared according to the synthetic sequences illustrated in Scheme 5.

Wherein
P and Q each independently is aryl or heteroaryl as described above
X is CH2, O, S
B represents —C(═O)—C₀-C₂-alkyl-.

The oxadiazole ring described below is prepared following synthetic routes well known in the art (Katrizky A. R. and Rees W. C. (1984) Comprehensive Heterocyclic Chemistry, Pergamon Press).

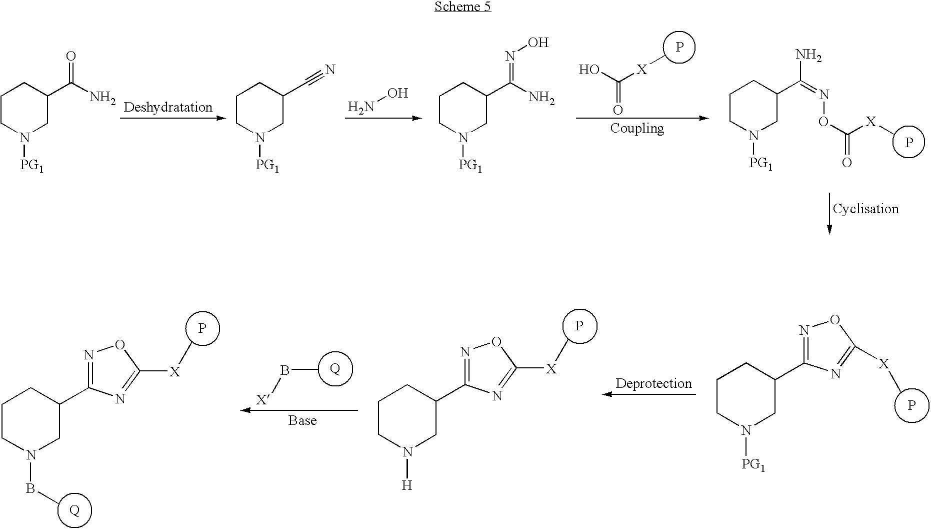

The starting nitrile derivative, prepared as described in Eur. J. Med. Chem., 1984, 19, 181-186, is reacted with hydroxylamine under neutral or basic conditions such as triethylamine, diisopropyl-ethylamine, sodium carbonate, sodium hydroxide and the like in a suitable solvent (e.g. methyl alcohol, ethyl alcohol). The reaction typically proceeds by allowing the reaction temperature to warm slowly from ambient temperature to a temperature range of 70° C. up to 80° C. inclusive for a time in the range of about 1 hour up to 48 hours inclusive (see for example Lucca, George V. De; Kim, Ui T.; Liang, Jing; Cordova, Beverly; Klabe, Ronald M.; et al; J. Med. Chem.; EN; 41; 13; 1998; 2411-2423, Lila, Christine; Gloanec, Philippe; Cadet, Laurence; Herve, Yolande; Fournier, Jean; et al.; Synth. Commun.; EN; 28; 23; 1998; 4419-4430 and see: Sendzik, Martin; Hui, Hon C.; Tetrahedron Lett.; EN; 44; 2003; 8697-8700 and references therein for reaction under neutral conditions).

The substituted amidoxime derivative (described in the Scheme 5) may be converted to an acyl-amidoxime derivative using the approach outlined in the Scheme 1. In the Scheme 1, PG₁ is an amino protecting group such as tert-Butyloxycarbonyl, benzyloxycarbonyl, ethoxycarbonyl, benzyl and the like. The coupling reaction may be promoted by coupling agents known in the art of organic synthesis such as EDCI (1-(3-dimethylaminopropyl)-3-ethylcarbodiimide), DCC(N,N′-dicyclohexyl-carbodiimide), in the presence of a suitable base such as triethylamine, diisopropyl-ethylamine, in a suitable solvent (e.g. tetrahydrofuran, dichloromethane, N,N-dimethylformamide, dioxane). Typically, a co-catalyst such as HOBT (hydroxy-benzotriazole), HOAT (1-hydroxy-7-azabenzotriazole) may also be present in the reaction mixture. The reaction typically proceeds at a temperature in the range of ambient temperature up to 60° C. inclusive for a time in the range of about 2 hours up to 12 hours to produce the intermediate acyl-amidoxime. The cyclisation reaction may be effected thermally in a temperature range of about 80° C. up to about 150° C. for a time in the range of about 2 hours up to 18 hours (see for example Suzuki, Takeshi; Iwaoka, Kiyoshi; Imanishi, Naoki; Nagakura, Yukinori; Miyata, Keiji; et al., Chem. Pharm. Bull., EN, 47: 1, 1999, 120-122). The product from the reaction can be isolated and purified employing standard techniques, such as extraction, chromatography, crystallization, distillation, and the like.

Then, the protecting group PGI is removed using standard methods. In the Scheme 5, B is as defined above, X′ is halogen or hydroxyl; for example the piperidine derivative is reacted with an aryl or heteroaryl acyl chloride using method that is readily apparent to those skilled in the art. The reaction may be promoted by a base such as triethylamine, diisopropylamine, pyridine in a suitable solvent (e.g. tetrahydrofuran, dichloromethane). The reaction typically proceeds by allowing the reaction temperature to warm slowly from 0° C. up to ambient temperature for a time in the range of about 4 up to 12 hours.

When X is OH, the coupling reaction may be promoted by coupling agents known in the art of organic synthesis such as EDCI (1-(3-Dimethylaminopropyl)-3-ethylcarbodiimide), DCC(N,N′-Dicyclohexyl-carbodiimide) or by polymer-supported coupling agents such as polymer-supported carbodiimide (PS-DCC, ex Argonaut Technologies), in the presence of a suitable base such as triethylamine, diisopropyl-ethylamine, in a suitable solvent (e.g. tetrahydrofuran, dichloromethane, N,N-dimethylformamide, dioxane). Typically, a co-catalyst such as HOBT (1-Hydroxy-benzotriazole), HOAT (1-Hydroxy-7-azabenzotriazole) and the like may also be present in the reaction mixture. The reaction typically proceeds at ambient temperature for a time in the range of about 2 hours up to 12 hours.

The compounds of formula III-A and IV-A wherein W is a 3-substituted piperidine ring may be prepared according to the synthetic sequences illustrated in the Scheme 6.

Wherein
P and Q each independently is aryl or heteroaryl as described above
X is CH2, O, S
B represents —C(═O)—C₀-C₂-alkyl-.

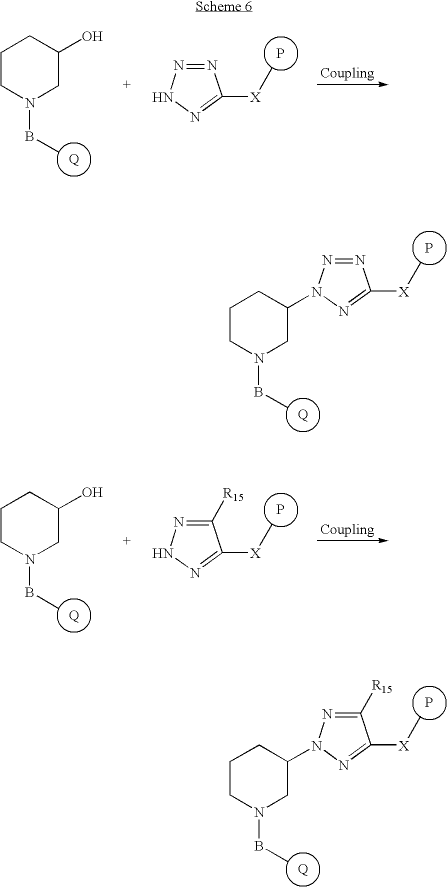

The precursor aryl-X-tetrazole derivatives are prepared according to synthetic routes well known in the art (Katrizky A. R. and Rees W. C. (1984) Comprehensive Heterocyclic Chemistry, Pergamon Press).

Aryl-X-tetrazole can be alkylated with a 3-hydroxypiperidine derivative under Mitsunobu coupling conditions, as described in the literature (see for example: Synthetic Commun; 26; 14; 1996; 2687-2694).

The compounds of Formula I which are basic in nature can form a wide variety of different pharmaceutically acceptable salts with various inorganic and organic acids. These salts are readily prepared by treating the base compounds with a substantially equivalent amount of the chosen mineral or organic acid in a suitable organic solvent such as methanol, ethanol or isopropanol (see Stahl P. H., Wermuth C. G., Handbook of Pharmaceuticals Salts, Properties, Selection and Use, Wiley, 2002).

The following non-limiting examples are intending to illustrate the invention. The physical data given for the compounds exemplified is consistent with the assigned structure of those compounds.

### EXAMPLES

Unless otherwise noted, all starting materials were obtained from commercial suppliers and used without further purification.

Specifically, the following abbreviation may be used in the examples and throughout the specification.

| | |
|---|---|
| g (grams) | RT (retention time) |
| mg (milligrams) | MeOH (methanol) |
| mL (millilitres) | EtOH (ethanol) |
| μl (microliters) | Hz (Hertz) |
| M (molar) | LCMS (Liquid Chromatography |
| | Mass Spectrum) |
| MHz (megahertz) | HPLC (High Pressure Liquid |
| | Chromatography) |
| mmol (millimoles) | NMR (Nuclear Magnetic |
| | Resonance) |
| min (minutes) | 1H (proton) |
| AcOEt (ethyl acetate) | Na₂SO₄ (sodium sulphate) |
| K₂CO₃ (potassium carbonate) | MgSO₄ (magnesium sulphate) |
| PdCl₂(PPh₃)₂ (Bis(triphenylphosphine) | |
| palladium (II) dichloride | |
| CDCl₃ (deutered chloroform) | HOBT (1-hydroxybenzotriazole) |
| EDCI•HCl (1- | r.t. (Room Temperature) |
| 3(Dimethylaminopropyl)-3- | |
| ethylcarbodiimide, hydrochloride) | |
| EtOH (ethyl alcohol) | NaOH (sodium hydroxide) |
| % (percent) | h (hour) |
| DCM (dichloromethane) | HCl (hydrochloric acid) |
| DIEA (diisopropyl ethyl amine) | n-BuLi (n-butyllithium) |
| Mp (melting point) | THF (tetrahydrofuran) |

All references to brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Brucker 300 MHz. Chemical shifts are expressed in parts of million (ppm, δ units). Coupling constants are in units of hertz (Hz) Splitting patterns describe apparent multiplicities and are designated as s (singlet), d (doublet), t (triplet), q (quadruplet), quint (quintuplet), m (multiplet).

Method A) Waters Alliance 2795 HT Micromass ZQ. Column Waters XTerra MS C18 (50×4.6 mm, 2.5 μm). Flow rate 1 ml/min Mobile phase: A phase=water/CH₃CN 95/5+0.05% TFA, B phase=water/CH₃CN=5/95+0.05% TFA. 0-1 min (A: 95%, B: 5%), 1-4 min (A: 0%, B: 100%), 4-6 min (A: 0%, B: 100%), 6-6.1 min (A: 95%, B: 5%). T=35° C.; UV detection: Waters Photodiode array 996, 200400 nm.

Method B) Pump 515, 2777 Sample Manager, Micromass ZQ Single quadrupole (Waters). Column 2.1*50 mm stainless steel packed with 3.5 μm SunFire RP C-18 (Waters); flow rate 0.25 ml/min splitting ratio MS:waste/1:4; mobile phase: A phase=water/acetonitrile 95/5+0.1% TFA, B phase=water/acetonitrile 5/95+0.1% TFA. 0-1.0 min (A: 98%, B: 2%), 1.0-5.0 min (A: 0%, B: 100%), 5.0-9.0 min (A: 0%, B: 100%), 9.01-12 min (A: 98%, B: 2%); UV detection wavelength 254 nm; Injection volume: 5 μl.

Method C): Pump 1525u (Waters), 2777 Sample Manager, Micromass ZQ2000 Single quadrupole (Waters); PDA detector: 2996 (Waters). Column 2.1*30 mm stainless steel packed with 3.0 μm Luna C18; flow rate 0.25 ml/min splitting ratio MS:waste/1:4; mobile phase: A phase=water/acetonitrile 95/5+0.1% TFA, B phase=water/acetonitrile 5/95+0.1% TFA. 0-1.0 min (A: 98%, B: 2%), 1.0-5.0 min (A: 0%, B: 100%), 5.0-9.0 min (A: 0%, B: 100%), 9.1-12 min (A: 98%, B: 2%); UV detection wavelength 254 nm; Injection volume: 5 μl.

Method D) Waters Alliance 2795 HT Micromass ZQ. Column Waters Symmetry C18 (75×4.6 mm, 3.5 μm). Flow rate 1.5 ml/min. Mobile phase: A phase=water/CH₃CN 95/5+0.05% TFA, B phase=water/CH₃CN=5/95+0.05% TFA.

0-2 min (A: 95%, B: 5%), 6 min (A: 0%, B: 100%), 6-8 min (A: 0%, B: 100%), 8-8.1 min (A: 95%, B: 5%). T=35° C.; UV detection: Waters Photodiode array 996, 200-400 nm.

Method E) Waters Alliance 2795 HT Micromass ZQ. Column Waters Symmetry C18 (75×4.6 mm, 3.5 μm). Flow rate 1.5 ml/min. Mobile phase: A phase=water/CH₃CN 95/5+0.05% TFA, B phase=water/CH₃CN=5/95+0.05% TFA.

0-0.5 min (A: 95%, B: 5%), 0.5-7 min (A: 0%, B: 100%), 7-8 min (A: 0%, B: 100%), 8-8.1 min (A: 95%, B: 5%). T=35° C.; UV detection: Waters Photodiode array 996, 200-400 nm.

Method F): HPLC system: Waters Acquity, MS detector: Waters ZQ2000. Column: Acquity HPLC-BEH C18 50×2.1 mm×1.7 um; flow rate 0.4 ml/min; mobile phase: A phase=water/acetonitrile 95/5+0.1% TFA, B phase=water/acetonitrile 5/95+0.1% TFA. 0-0.25 min (A: 98%, B: 2%), 0.25-4.0 min (A: 0%, B: 100%), 4.0-5.0 min (A: 0%, B: 100%), 5.1-6 min (A: 98%, B: 2%); UV detection wavelength 254 nm.

Method G): HPLC system: Waters Acquity, MS detector: Waters ZQ2000. Column: Acquity HPLC-BEH C18 50×2.1 mm×1.7 um; flow rate 0.6 ml/min; mobile phase: A phase=water/acetonitrile 95/5+0.1% TFA, B phase=water/acetonitrile 5/95+0.1% TFA. 0-0.25 min (A: 98%, B: 2%), 3.30 min (A: 0%, B: 100%), 3.3-4.0 min (A: 0%, B: 100%), 4.1 min (A: 98%, B: 2%); UV detection wavelength 254 nm.

Method H): HPLC system: Waters Acquity, MS detector: Waters ZQ2000. Column: Acquity HPLC-BEH C18 50×2.1 mm×1.7 um; flow rate 0.25 ml/min; mobile phase: A phase=water/acetonitrile 95/5+0.1% TFA, B phase=water/acetonitrile 5/95+0.1% TFA. 0-1.0 min (A: 98%, B: 2%), 1.0-5.0 min (A: 0%, B: 100%), 5.0-9.0 min (A: 0%, B: 100%), 9.1-12 min (A: 98%, B: 2%); UV detection wavelength 254 nm.

Method 1) Waters Alliance 2795 HT Micromass ZQ. Column Waters Symmetry C18 (75×4.6 mm, 3.5 μm). Flow rate 1.5 ml/min. Mobile phase: A phase=water/CH₃CN 95/5+0.05% TFA, B phase=water/CH₃CN=5/95+0.05% TFA.

0-0.1 min (A: 95%, B: 5%), 6 min (A: 0%, B: 100%), 6-8 min (A: 0%, B: 100%), 8.1 min (A: 95%, B: 5%). T=35° C.; UV detection: Waters Photodiode array 996, 200-400 nm.

Method L) Waters Alliance 2795 HT Micromass ZQ. Column Waters XTerra MS C18 (50×4.6 mm, 2.5 μm). Flow rate 1.2 ml/min Mobile phase: A phase=water/CH₃CN 95/5+0.05% TFA, B phase=water/CH₃CN=5/95+0.05% TFA. 0-0.8 min (A: 95%, B: 5%), 3.3 min (A: 0%, B: 100%), 3.3-5 min (A: 0%, B: 100%), 5.1 min (A: 95%, B: 5%). T=35° C.; UV detection: Waters Photodiode array 996, 200-400 nm.

Method M): HPLC system: Waters Acquity, MS detector: Waters ZQ2000. Column: Acquity HPLC-BEH C18 50×2.1 mm×1.7 um; flow rate 0.5 ml/min; mobile phase: A phase=water/acetonitrile 95/5+0.1% TFA, B phase=water/acetonitrile 5/95+0.1% TFA. 0-0.1 min (A: 95%, B: 5%), 1.6 (A: 0%, B: 100%), 1.6-1.9 min (A: 0%, B: 100%), 2.4 min (A: 95%, B: 5%); UV detection wavelength 254 nm.

All mass spectra were taken under electrospray ionisation (ESI) methods.

The microwave oven used is an apparatus from Biotage (Optimizer™) equipped with an internal probe that monitors reaction temperature and pressure, and maintains the desired temperature by computer control.

Most of the reactions were monitored by thin-layer chromatography on 0.25 mm Macherey-Nagel silica gel plates (60F-2254), visualized with UV light. Flash column chromatography was performed on silica gel (220-440 mesh, Fluka). Melting point determination was performed on a Buchi B-540 apparatus.

### Example 1

### {(S)-3-[3-(4-Fluoro-benzyl)-[1,2,4]oxadiazol-5-yl]-piperidin-1-yl}-(4-fluoro-phenyl)-methanone

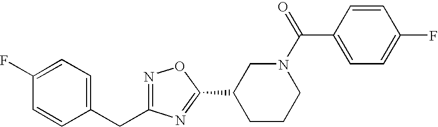

### 1(A) (S)-3-[3-(4-Fluoro-benzyl)-[1,2,4]oxadiazol-5-yl]-piperidine-1-carboxylic acid tert-butyl ester

To a solution of 4-fluorophenylacetonitrile (0.37 mL, 3 mmol) in EtOH (4 mL), hydroxylamine (50% wt. aqueous solution, 0.74 mL, 12 mmol) was added at room temperature and the solution was stirred under reflux for 1.5 h. The solvent was removed under reduced pressure to afford 2-(4-fluoro-phenyl)-N-hydroxy-acetamidine that was used immediately for the next step.

A mixture of 2-(4-fluoro-phenyl)-N-hydroxy-acetamidine (3 mmol), S-1-Boc-piperidine-3-carboxylic acid (0.69 g, 3 mmol), EDCI.HCl (0.86 g, 4.5 mmol), HOBT (0.46 g, 3 mmol) and TEA (0.84 mL, 6 mmol) in dioxane (10 mL) was stirred for 24 h at room temperature, under nitrogen atmosphere, then the reaction mixture was heated under reflux for 8 h. The solvent was evaporated under reduced pressure. The residue was diluted with water (50 mL) and ethyl acetate (50 mL), the phases were separated and the organic layer was washed sequentially with water (50 mL×2 times) and with NaOH 1N (50 mL×2 times). The organic layer was dried over Na₂SO₄ and concentrated under reduced pressure. Purification of the crude by flash chromatography (silica gel, eluent: DCM/MeOH/NH₄OH 99.5/0.5/0.05) gave 0.74 g of (S)-3-[3-(4-Fluoro-benzyl)-[1,2,4]oxadiazol-5-yl]-piperidine-1-carboxylic acid tert-butyl ester.

Yield: 68%; LCMS (RT): 5.5 min (Method A); MS (ES+) gave m/z: 362.1.

¹H-NMR (DMSO-d₆, 363 K), δ (ppm): 7.34 (dd, 2H); 7.09 (dd, 2H); 4.06 (s, 2H); 3.97 (m, 1H); 3.63 (m, 1H); 3.34 (dd, 1H); 3.20-3.05 (m, 2H); 2.10 (m, 1H); 1.83 (m, 1H); 1.71 (m, 1H); 1.59-1.44 (m, 1H); 1.40 (s, 9H).

### 1(B) (S)-3-[3-(4-Fluoro-benzyl)-[1,2,4]oxadiazol-5-yl]-piperidine hydrochloride

(S)-3-[3-(4-Fluoro-benzyl)-[1,2,4]oxadiazol-5-yl]-piperidine-1-carboxylic acid tert-butyl ester (0.73 g, 2 mmol) was dissolved in dioxane (2 mL) and 4 mL of HCl 4N (dioxane solution) were added dropwise at 0° C. The resulting mixture was stirred at room temperature for 1.5 h. The solvent was evaporated under reduced pressure to afford 594 mg (yield: 100%) of (S)-3-[3-(4-Fluoro-benzyl)-[1,2,4]oxadiazol-5-yl]-piperidine hydrochloride as a white solid.

LCMS (RT): 3.67 min (Method A); MS (ES+) gave m/z: 262.1.

### 1(C) {(S)-3-[3-(4-Fluoro-benzyl)-[1,2,4]oxadiazol-5-yl]-piperidin-1-yl}-(4-fluoro-phenyl)-methanone

To a suspension of (S)-3-[3-(4-fluoro-benzyl)-[1,2,4]oxadiazol-5-yl]-piperidine hydrochloride (594 mg, 2 mmol) in dry dichloromethane (15 mL), triethylamine (0.7 mL, 5 mmol) and 4-fluorobenzoyl chloride (0.27 mL, 2.2 mmol) were added dropwise at 0° C. The reaction mixture was allowed to warm at room temperature and stirred for 24 h under nitrogen atmosphere. The solution was then treated with NaOH 1N (10 mL) and the phases were separated. The organic layer was washed with water (5 mL) and with brine (5 mL), then was dried over Na₂SO₄ and evaporated under reduced pressure. The crude was purified by flash chromatography (silica gel, eluent: DCM/MeOH/NH₄OH 99:1:0.1) to give 330 mg of the title compound.

Yield: 43% (pale brown oil); [α]_{D}²⁰=+74.2 (c=0.97, CHCl₃); LCMS (RT): 7.29 min (Method B); MS (ES+) gave m/z: 384.1.

¹H-NMR (DMSO-d₆), δ (ppm): 7.40 (dd, 2H); 7.33 (dd, 2H); 7.19 (dd, 2H); 7.09 (dd, 2H); 4.11 (dd, 1H); 4.07 (s, 2H); 3.70 (ddd, 1H); 3.49 (dd, 1H); 3.30 (m, 2H); 2.17 (m, 1H); 1.91 (m, 1H); 1.76 (m, 1H); 1.61 (m, 1H)

### Example 2

### (3,4-Difluoro-phenyl)-{(S)-3-[3-(4-fluoro-benzyl)-[1,2,4]oxadiazol-5-yl]-piperidin-1-yl}-methanone

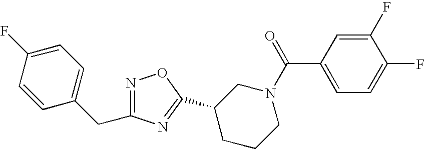

The title compound was obtained following the same procedure described in Example 1(C), starting from (S)-3-[3-(4-fluoro-benzyl)-[1,2,4]oxadiazol-5-yl]-piperidine hydrochloride (prepared as described in Example 1(B)) and 3,4-difluorobenzoyl chloride. Purification by flash chromatography (silica gel, eluent: DCM/MeOH/NH₄OH 99.5:0.5:0.05) and successive trituration from diethyl ether gave 80 mg of (3,4-difluoro-phenyl)-{(S)-3-[3-(4-fluoro-benzyl)-[1,2,4]oxadiazol-5-yl]-piperidin-1-yl}-methanone.

Yield: 29% (white powder); [α]_{D}²⁰=+64.22 (c=0.86, MeOH); LCMS (XD: 6.76 min (Method C); MS (ES+) gave m/z: 402.2 (MH+).

¹H-NMR (DMSO-d₆, 343K), δ (ppm): 7.49-7.29 (m, 4H); 7.20 (m, 1H); 7.10 (dd, 2H); 4.09 (m, 1H); 4.07 (s, 2H); 3.67 (m, 1H); 3.48 (dd, 1H); 3.37-3.23 (m, 2H); 2.16 (m, 1H); 1.89 (m, 1H); 1.73 (m, 1H); 1.60 (m, 1H)

### Example 3

### (3,4-Difluoro-phenyl)-{(S)-3-[5-(4-fluoro-benzyl)-[1,2,4]oxadiazol-3-yl]-piperidin-1-yl}-methanone

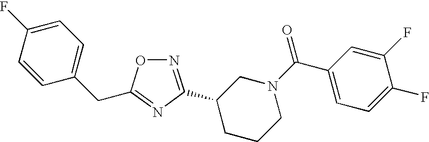

### 3 (A) (S)-3-Carbamoyl-piperidine-1-carboxylic acid tert-butyl ester

Triethylamine (1.21 mL, 8.72 mmol) and then ethyl chloroformate (0.8 mL, 8.30 mmol) were added dropwise at 0° C. to a solution of (S)-1-Boc-piperidine-3-carboxylic acid (2 g, 8.72 mmol) in chloroform (40 mL), under nitrogen atmosphere. After stirring 10 min at 0° C., NH₃ (gas) was bubbled into the solution for 1 h. The reaction mixture was then stirred at room temperature for 3 h, 5% NaHCO₃ (aq) was added and the phases were separated. The organic layer was dried over sodium sulphate and evaporated under reduced pressure to afford the title compound, which was used for the next step without further purification.

Yield: quantitative; LCMS (RT): 3.31 min (Method A); MS (ES+) gave m/z: 229.0.

### 3 (B) (S)-3-Cyano-piperidine-1-carboxylic acid tert-butyl ester

Phosphorus oxychloride (812 uL, 8.72 mmol) was added dropwise at 0° C. to a solution of (S)-3-carbamoyl-piperidine-1-carboxylic acid tert-butyl ester (2 g, 8.72 mmol) in pyridine (20 mL), under nitrogen atmosphere. After stirring overnight at room temperature, ethyl acetate was added and the solution was washed with 10% HCl (2 times). The phases were separated and the organics were dried over sodium sulphate and evaporated to dryness under reduced pressure.

The title compound was used for the next step without further purification.

Yield: quantitative; LCMS (RT): 4.48 min (Method A); MS (ES+) gave m/z: 211.1.

### 3 (C) (S)-3-(N-Hydroxycarbamimidoyl)-piperidine-1-carboxylic acid tert-butyl ester

A solution of (S)-3-cyano-piperidine-1-carboxylic acid tert-butyl ester (1.8 g, 8.72 mmol) and aqueous hydroxylamine (50% in water, 2.1 mL, 34.88 mmol) in ethanol (20 mL) was refluxed for 2 h. The solvent was evaporated under reduced pressure to afford the title compound that was used for the next step without further purification.

Yield: quantitative; LCMS (RT): 2.71 min (Method A); MS (ES+) gave m/z: 244.0.

### 3 (D) (S)-3-[5-(4-Fluoro-benzyl)-[1,2,4]oxadiazol-3-yl]-piperidine-1-carboxylic acid tert-butyl ester

A mixture of (S)-3-(N-hydroxycarbamimidoyl)-piperidine-1-carboxylic acid tert-butyl ester (360 mg, 1.48 mmol), 4-fluorophenylacetic acid (0.230 g, 1.48 mmol), HOBT (0.2 g, 2.22 mmol), EDCI.HCl (0.429 g, 1.48 mmol) and dry triethylamine (0.41 mL, 2.96 mmol) in dry dioxane (10 mL) was kept under stirring at ambient temperature for 12 h, under nitrogen atmosphere. The reaction mixture was then refluxed for 4 h and the solvent was evaporated under reduced pressure. The residue was diluted with water (40 mL) and ethyl acetate (40 mL), the phases were separated and the organic layer was washed sequentially with 5% citric acid (40 mL), water (40 mL, twice), Na₂CO₃ 1N (40 mL, twice) and with brine. The organic layer was dried over sodium sulphate and the solvent was removed under vacuum to give a residue that was purified by flash chromatography (silica gel, eluent: hexane/ethyl acetate 85:15) to give the pure title compound (105 mg).

Yield: 20%; LCMS (RT): 5.5 min (Method I); MS (ES+) gave m/z: 362.04.

### 3 (E) (S)-3-[5-(4-Fluoro-benzyl)-[1,2,4]oxadiazol-3-yl]-piperidine hydrochloride

(S)-3-[5-(4-Fluoro-benzyl)-[1,2,4]oxadiazol-3-yl]-piperidine-1-carboxylic acid tert-butyl ester (0.105 g, 0.29 mmol) was dissolved in dioxane (2 mL) and 4 mL of HCl 4N (dioxane solution) were added dropwise at 0° C. The resulting mixture was stirred at room temperature for 1.5 h. The solvent was evaporated under reduced pressure to afford 86 mg (yield: 100%) of (S)-3-[5-(4-Fluoro-benzyl)-[1,2,4]oxadiazol-3-yl]-piperidine hydrochloride as a white solid.

LCMS (RT): 3.9 min (Method D); MS (ES+) gave m/z: 262.1.

### 3 (F) (3,4-Difluoro-phenyl)-{(S)-3-[5-(4-fluoro-benzyl)-[1,2,4]oxadiazol-3-yl]-piperidin-1-yl}-methanone

To a suspension of (S)-3-[5-(4-fluoro-benzyl)-[1,2,4]oxadiazol-3-yl]-piperidine hydrochloride (86 mg, 0.29 mmol) in dry dichloromethane (8 mL), triethylamine (0.1 mL, 0.73 mmol) and 3,4-difluorobenzoyl chloride (0.042 mL, 0.34 mmol) were added dropwise at 0° C. The reaction mixture was allowed to warm at room temperature and stirred for 24 h under nitrogen atmosphere. The solution was then treated with NaOH 1N (10 mL) and the phases were separated. The organic layer was washed with water (5 mL) and with brine (5 mL), then was dried over Na₂SO₄ and evaporated under reduced pressure. The crude was purified by flash chromatography (silica gel, eluent: DCM/MeOH/NH₄OH 99:1:0.1) to give 45 mg of the title compound.

Yield: 39% (colourless gummy solid); [α]_{D}²⁰+36.57 (c=0.90, MeOH); LCMS (RT): 6.98 min (Method C); MS (ES+) gave m/z: 402.1 (MH+).

¹H-NMR (DMSO-d₆, 343K), δ (ppm): 7.49-7.32 (m, 4H); 7.23 (m, 1H); 7.14 (dd, 2H); 4.31 (s, 2H); 4.11 (m, 1H); 3.77 (m, 1H); 3.30 (dd, 1H); 3.19 (ddd, 1H); 3.08 (m, 1H); 2.12 (m, 1H); 1.88-1.71 (m, 2H); 1.61 (m, 1H).

### Example 4

### {(S)-3-[5-(4-Fluoro-benzyl)-[1,2,4]oxadiazol-3-yl]-piperidin-1-yl}-(4-fluoro-phenyl)-methanone

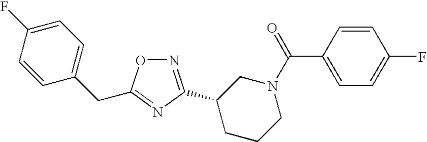

The title compound was obtained following the same procedure described in Example 3(F), starting from (S)-3-[5-(4-fluoro-benzyl)-[1,2,4]oxadiazol-3-yl]-piperidine hydrochloride (prepared as described in Example 3(E)) and 4-fluorobenzoyl chloride. Purification by flash chromatography (silica gel, eluent: DCM/MeOH/NH₄OH 99.5:0.5:0.05) gave 20 mg of {(S)-3-[5-(4-Fluoro-benzyl)-[1,2,4]oxadiazol-3-yl]-piperidin-1-yl}-(4-fluoro-phenyl)-methanone.

Yield: 18% (Colourless oil); [α]_{D}²⁰=+44.53 (c=0.76, MeOH); LCMS (RT): 6.83 min (Method C); MS (ES+) gave m/z: 384.1 (MH+).

¹H-NMR (DMSO-d₆, 343K), δ (ppm): 7.43 (dd, 2H); 7.37 (dd, 2H); 7.2 1 (dd, 2H); 7.14 (dd, 2H); 4.31 (s, 2H); 4.15 (m, 1H); 3.79 (m, 1H); 3.29 (dd, 1H); 3.18 (ddd, 1H); 3.06 (m, 1H); 2.12 (m, 1H); 1.88-1.72 (m, 2H); 1.59 (m, 1H)

### Example 5

### (4-Fluoro-phenyl)-{(S)-3-[5-((S)-1-phenyl-ethyl)-[1,2,4]oxadiazol-3-yl]-piperidin-1-yl}-methanone

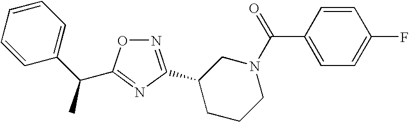

### 5 (A) (S)-1-(4-Fluoro-benzoyl)-piperidine-3-carbonitrile

(S)-3-Cyano-piperidine-1-carboxylic acid tert-butyl ester (1.5 g, 7.14 mmol), prepared as described in Example 3(B), was dissolved in dioxane (15 mL) and 10 mL of HCl 4N (dioxane solution) were added dropwise at 0° C. The resulting mixture was stirred at room temperature for 5 h. The solvent was evaporated under reduced pressure to afford (S)-piperidine-3-carbonitrile hydrochloride as a white solid, that was used for the next step without further purification.

To a suspension of (S)-piperidine-3-carbonitrile hydrochloride (7.14 mmol) in dry dichloromethane (100 mL), triethylamine (3 mL, 21.4 mmol) and 4-fluorobenzoyl chloride (930 uL, 7.85 mmol) were added dropwise at 0° C. The reaction mixture was allowed to warm at room temperature and stirred for 3 h under nitrogen atmosphere. The solution was then treated with 5% NaHCO₃ (50 mL, twice) and the phases were separated. The organic layer was washed with 1N HCl (50 mL) and with brine (50 mL), then was dried over Na₂SO₄ and evaporated under reduced pressure. The crude was purified by flash chromatography (silica gel, eluent gradient: from petroleum ether/ethyl acetate 7:3 to petroleum ether/ethyl acetate 1:1) to give 1.01 g of the title compound.

Yield: 61% (yellow oil); LCMS (RT): 3.7 min (Method E); MS (ES+) gave m/z: 233.1.

### 5 (B) (S)-1-(4-Fluoro-benzoyl)-N-hydroxy-piperidine-3-carboxamidine

A solution of (S)-1-(4-fluoro-benzoyl)-piperidine-3-carbonitrile (1.01 g, 4.35 mmol) and aqueous hydroxylamine (50% in water, 1.1 mL, 17.4 mmol) in ethanol (10 mL) was refluxed for 4 h. The solvent was evaporated under reduced pressure to afford the title compound (1.15 g) that was used for the next step without further purification.

Yield: quantitative; ¹H-NMR (DMSO-d₆, 343K), δ (ppm): 8.61 (s br, 1H); 7.44 (dd, 2H); 7.22 (dd, 2H); 5.12 (s br, 2H); 4.00 (m, 2H); 3.17-2.82 (m, 3H); 2.23 (m, 1H); 1.98 (m, 1H); 1.78-1.55 (m, 2H).

### 5 (C) (4-Fluoro-phenyl)-{(S)-3-[5-((S)-1-phenyl-ethyl)-[1,2,4]oxadiazol-3-yl]-piperidin-1-yl}-methanone

A mixture of (S)-1-(4-Fluoro-benzoyl)-N-hydroxy-piperidine-3-carboxamidine (200 mg, 0.75 mmol), (S)-2-phenylpropionic acid (0.12 mL, 0.83 mmol), HOAT (0.1 g, 0.75 mmol), EDCI.HCl (0.22 g, 1.13 mmol) and dry triethylamine (0.21 mL, 1.51 mmol) in dry dioxane (10 mL) was kept under stirring at ambient temperature for 24 h, under nitrogen atmosphere. The reaction mixture was then refluxed for 6 h and the solvent was evaporated under reduced pressure. The residue was diluted with water (40 mL) and ethyl acetate (40 mL), the phases were separated and the organic layer was washed sequentially with 5% citric acid (40 mL), water (40 mL, twice), Na₂CO₃ 1N (40 mL, twice) and with brine. The organic layer was dried over sodium sulphate and the solvent was removed under vacuum to give a residue that was purified by flash chromatography (silica gel, eluent: DCM/MeOH/NH₄OH 99.6:0.4:0.04) to give the pure title compound (180 mg).

Yield: 63% (Colourless oil); [α]_{D}²⁰=+93.6 (c=1.06, MeOH); LCMS (RT): 8.37 min (Method C); MS (ES+) gave m/z: 380.2 (MH+).

¹H-NMR (DMSO-d₆, 343K), δ (ppm): 7.50-7.14 (m, 9H); 4.53 (q, 1H); 4.15 (m, 1H); 3.77 (m, 1H); 3.32 (dd, 1H); 3.19 (ddd, 1H); 3.07 (m, 1H); 2.13 (m, 1H); 1.91-1.71 (m, 2H); 1.69-1.49 (m, 1H); 1.66 (d, 3H).

### Example 6

### (4-Fluoro-phenyl)-{(S)-3-[5-((R)-1-phenyl-ethyl)-[1,2,4]oxadiazol-3-yl]-piperidin-1-yl}-methanone

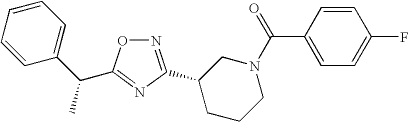

The title compound was obtained following the same procedure described in Example 5(C), starting from (S)-1-(4-fluoro-benzoyl)-N-hydroxy-piperidine-3-carboxamidine (prepared as described in Example 5(B)) and (R)-2-phenylpropionic acid. Purification by flash chromatography (silica gel, eluent: DCM/MeOH/NH₄OH 99.6:0.4:0.04) gave 90 mg of (4-Fluoro-phenyl)-{(S)-3-[5-((R)-1-phenyl-ethyl)-[1,2,4]oxadiazol-3-yl]-piperidin-1-yl}-methanone.

Yield: 42% (Colourless gummy solid); [α]_{D}²⁰=+30.7 (c=0.96, MeOH); LCMS (RT): 7.07 min (Method C); MS (ES+) gave m/z: 380.2 (MH+).

¹H-NMR (DMSO-d₆, 343K), δ (ppm): 7.43 (dd, 2H); 7.38-7.27 (m, 5H); 7.21 (dd, 2H); 4.53 (q, 1H); 4.16 (m, 1H); 3.77 (m, 1H); 3.32 (dd, 1H); 3.21 (ddd, 1H); 3.07 (m, 1H); 2.13 (m, 1H); 1.90-1.73 (m, 2H); 1.66 (d, 3H); 1.60 (m, 1H)

### Example 7

### [(S)-3-(5-Benzyl-[1,2,4]oxadiazol-3-yl)-piperidin-1-yl]-(4-fluoro-phenyl)-methanone

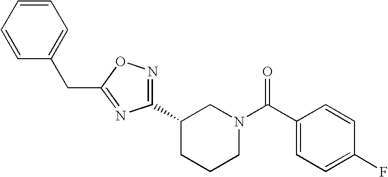

The title compound was obtained following the same procedure described in Example 5(C), starting from (S)-1-(4-Fluoro-benzoyl)-N-hydroxy-piperidine-3-carboxamidine (prepared as described in Example 5(B)) and phenylacetic acid. Purification by flash chromatography (silica gel, eluent: petroleum ether/acetone 8:1) and successive crystallization from petroleum ether/diethyl ether gave 56 mg of [(S)-3-(5-Benzyl-[1,2,4]oxadiazol-3-yl)-piperidin-1-yl]-(4-fluoro-phenyl)-methanone.

Yield: 27% (white solid); [ac]_{D}²⁰=+67.2 (c-0.99, MeOH); mp=75° C.; LCMS (RT): 6.82 min (Method C); MS (ES+) gave m/z: 366.2 (MH+).

¹H-NMR (DMSO-d₆, 343K), δ (ppm): 7.43 (dd, 2H); 7.38-7.26 (m, 5H); 7.21 (dd, 2H); 4.30 (s, 2H); 4.15 (m, 1H); 3.79 (m, 1H); 3.30 (dd, 1H); 3.19 (ddd, 1H); 3.06 (m, 1H); 2.13 (m, 1H); 1.88-1.72 (m, 2H); 1.59 (m, 1H).

### Example 8

### (4-Fluoro-phenyl)-{(S)-3-[5-((S)-hydroxy-phenyl-methyl)-[1,2,4]oxadiazol-3-yl]-piperidin-1-yl}-methanone

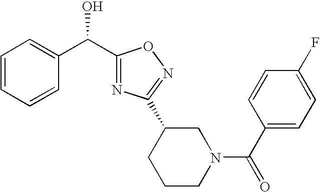

The title compound was obtained following the same procedure described in Example 5(C), starting from (S)-1-(4-Fluoro-benzoyl)-N-hydroxy-piperidine-3-carboxamidine (prepared as described in Example 5(B)) and (L)-mandelic acid. Purification by flash chromatography (silica gel, eluent: DCM/MeOH 96:4) and successive trituration from ethyl acetate/diethyl ether gave 22 mg of (4-Fluoro-phenyl)-{(S)-3-[5-((S)-hydroxy-phenyl-methyl)-[1,2,4]oxadiazol-3-yl]-piperidin-1-yl}-methanone.

Yield: 15% (White powder); [α]_{D}²⁰=+48.54 (c=0.56, MeOH); mp=168-172° C.;

LCMS (RT): 6.17 min (Method C); MS (ES+) gave m/z: 382.1 (MH+).

¹H-NMR (DMSO-d₆, 373K), δ (ppm): 7.49-7.30 (m, 7H); 7.18 (dd, 2H); 6.25 (d br, 1H); 5.98 (d, 1H); 4.14 (m, 1H); 3.77 (m, 1H); 3.33 (dd, 1H); 3.21 (m, 1H); 3.08 (m, 1H); 2.15 (m, 1H); 1.92-1.73 (m, 2H); 1.62 (m, 1H).

### Example 9

### (4-Fluoro-phenyl)-{(S)-3-[5-((R)-hydroxy-phenyl-methyl)-[1,2,4]oxadiazol-3-yl]-piperidin-1-yl}-methanone

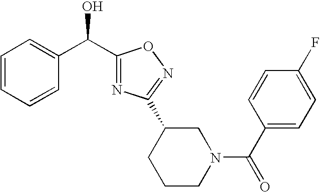

The title compound was obtained following the same procedure described in Example 5(C), starting from (S)-1-(4-Fluoro-benzoyl)-N-hydroxy-piperidine-3-carboxamidine (prepared as described in Example 5(B)) and (D)-mandelic acid. Purification by preparative HPLC gave 40 mg of (4-Fluoro-phenyl)-{(S)-3-[5-((R)-hydroxy-phenyl-methyl)-[1,2,4]oxadiazol-3-yl]-piperidin-1-yl}-methanone.

Yield: 9% (Pale yellow gummy solid); [α]_{D}²⁰=+46.35 (c=0.55, MeOH); LCMS (RT): 6.29 min (Method C); MS (ES+) gave m/z: 382.1.

¹H-NMR (DMSO-d₆, 368K), δ (ppm): 7.48-7.31 (m, 7H); 7.19 (dd, 2H); 5.99 (s, 1H); 4.16 (dd br, 1H); 3.80 (ddd br, 1H); 3.32 (dd, 1H); 3.20 (ddd, 1H); 3.07 (ddd, 1H); 2.14 (m, 1H); 1.90-1.73 (m, 2H); 1.60 (m, 1H).

### Example 10

### (4-Fluoro-phenyl)-[(S)-3-(5-phenethyl-[1,2,4]oxadiazol-3-yl)-piperidin-1-yl]-methanone

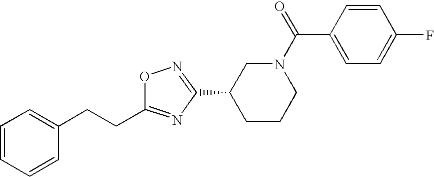

The title compound was obtained following the same procedure described in Example 5(C), starting from (S)-1-(4-fluoro-benzoyl)-N-hydroxy-piperidine-3-carboxamidine (prepared as described in Example 5(B)) and 3-phenylpropionic acid. Purification by flash chromatography (silica gel, eluent: hexane/ethyl acetate 8:2) and successive preparative HPLC gave 110 mg of (4-Fluoro-phenyl)-[(S)-3-(5-phenethyl-[1,2,4]oxadiazol-3-yl)-piperidin-1-yl]-methanone.

Yield: 22% (Pale yellow oil); [α]_{D}²⁰=+61.9 (c=0.88 MeOH); LCMS (RT): 7.12 min (Method C); MS (ES+) gave m/z: 380.2 (MH+).

¹H-NMR (DMSO-d₆, 343K), δ (ppm): 7.45 (dd, 2H); 7.30-7.15 (m, 7H); 4.15 (m, 1H); 3.82 (m, 1H); 3.31-3.12 (m, 4H); 3.09-2.98 (m, 3H); 2.12 (m, 1H); 1.87-1.71 (m, 2H); 1.60 (m, 1H)

### Example 11

### {3-[(S)-1-(4-Fluoro-benzoyl)-piperidin-3-yl]-[1,2,4]oxadiazol-5-yl}-phenyl-methanone

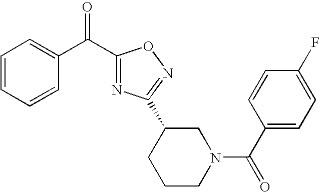

A mixture of (4-fluoro-phenyl)-{(S)-3-[5-((R)-hydroxy-phenyl-methyl)-[1,2,4]oxadiazol-3-yl]-piperidin-1-yl}-methanone (80 mg, 0.21 mmol), prepared as described in Example 9, manganese dioxide (96 mg, 0.95 mmol) in dry THF (10 mL) was stirred at room temperature for 12 h under nitrogen atmosphere. The solvent was evaporated and the residue was diluted with ethyl acetate and washed with water (20 mL). The organic layer was dried over sodium sulphate and evaporated under reduced pressure to give a crude residue that was purified by flash chromatography (silica gel, eluent: DCM/MeOH/NH₄OH 99:1:0.1).

{3-[(S)-1-(4-Fluoro-benzoyl)-piperidin-3-yl]-[1,2,4]oxadiazol-5-yl}-phenyl-methanone was obtained as an off-white powder (18 mg).

Yield: 23% (off-white powder); mp=80-83° C.; LCMS (RT): 6.97 min (Method C);

MS (ES+) gave m/z: 380.1 (MH+).

¹H-NMR (DMSO-d₆, 368K), δ (ppm): 8.25 (dd, 2H); 7.79 (dd, 1H); 7.63 (dd, 2H); 7.46 (dd, 2H); 7.19 (dd, 2H); 4.24 (dd br, 1H); 3.81 (ddd br, 1H); 3.50 (dd, 1H); 3.28 (m, 2H); 2.25 (m, 1H); 1.98 (m, 1H); 1.85 (m, 1H); 1.67 (m, 1H).

### Example 12

### (4-Fluoro-phenyl)-[(S)-3-(5-phenylamino-[1,2,4]oxadiazol-3-yl)-piperidin-1-yl]-methanone

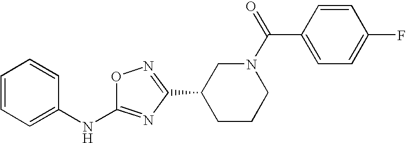

To a solution of (S)-1-(4-fluoro-benzoyl)-N-hydroxy-piperidine-3-carboxamidine (150 mg, 0.57 mmol), prepared as described in Example 5(B), in acetonitrile (3 mL), phenylisocyanate (63 uL, 0.57 mmol) and triethylamine (156 uL, 1.14 mmol) were added and the mixture was heated in a microwave oven at 150° C. for 15 min, in a sealed tube. BEMP (156 mg, 0.57 mmol) was then added and the mixture was heated in a microwave oven at 150° C. for 15 min, in a sealed tube. Another portion of phenylisocyanate (63 uL) and BEMP (100 uL) was added to the reaction mixture. After heating at 150° C. for 20 min in a microwave oven in a sealed tube, the solvent was removed under reduced pressure and the crude residue was purified by flash chromatography (silica gel, eluent: DCM/MeOH/NH₄OH 99:1:0.1).

(4-Fluoro-phenyl)-[(S)-3-(5-phenylamino-[1,2,4]oxadiazol-3-yl)-piperidin-1-yl]-methanone was obtained as a brown solid (30 mg).

Yield: 14% (brown solid); [α]_{D}²⁰=+20.26 (c=0.4, CH₃OH); LCMS (RT): 6.59 min (Method C); MS (ES+) gave m/z: 367.1 (MH+).

¹H-NMR (DMSO-d₆, 373K), δ (ppm): 10.33 (s br, 1H); 7.52 (dd, 2H); 7.45 (dd, 2H); 7.34 (dd, 2H); 7.17 (dd, 2H); 7.07 (dd, 1H); 4.17 (m, 1H); 3.84 (m, 1H); 3.33 (dd, 1H); 3.26-3.09 (m, 1H); 2.17 (m, 1H); 1.93-1.79 (m, 2H); 1.62 (m, 2H).

### Example 13

### {(S)-3-[5-(4-Fluoro-benzylamino)-[1,2,4]oxadiazol-3-yl]-piperidin-1-yl}-(4-fluoro-phenyl)-methanone

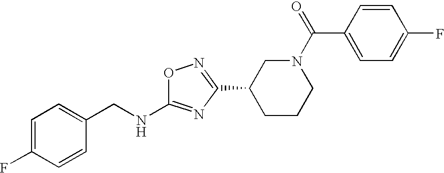

The title compound was obtained following the same procedure described in Example 12, starting from (S)-1-(4-fluoro-benzoyl)-N-hydroxy-piperidine-3-carboxamidine (prepared as described in Example 5(B)) and 4-fluorobenzyl isocyanate. Purification by flash chromatography (silica gel, eluent: DCM/MeOH 98:2) and successive preparative HPLC gave 40 mg of {(S)-3-[5-(4-Fluoro-benzylamino)-[1,2,4]oxadiazol-3-yl]-piperidin-1-yl}-(4-fluoro-phenyl)-methanone.

Yield: 13% (Pale yellow oil); LCMS (RT): 6.49 min (Method C); MS (ES+) gave m/z: 399.1 (MH+).

¹H-NMR (DMSO-d₆), δ (ppm): 8.47 (t br, 1H); 7.44 (dd, 2H); 7.35 (dd, 2H); 7.2 1 (dd, 2H); 7.12 (dd, 2H); 4.40 (d, 2H); 4.10 (m, 1H); 3.82 (m, 1H); 3.26-3.02 (m, 2H); 2.81 (m, 1H); 2.06 (m, 1H); 1.84-1.68 (m, 2H); 1.55 (m, 1H).

### Example 14

### [(S)-3-(5-Benzyl-tetrazol-2-yl)-piperidin-1-yl]-(4-fluoro-phenyl)-methanone

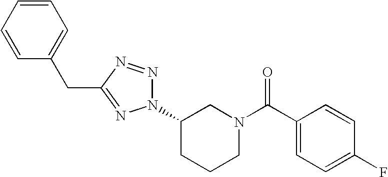

### 14 (A) (4-Fluoro-phenyl)-((R)-3-hydroxy-piperidin-1-yl)-methanone

A mixture of (R)-3-hydroxy-piperidine hydrochloride (200 mg, 1.45 mmol), 4-fluorobenzoic acid (204 mg, 1.45 mmol), HOBT (196 mg, 1.45 mmol), EDCI.HCl (420 mg, 2.18 mmol) and dry triethylamine (0.32 mL, 4.36 mmol) in dry DCM (10 mL) was kept under stirring at ambient temperature overnight, under nitrogen atmosphere. The mixture was treated sequentially with 0.1N HCl (40 mL), 0.1N NaOH (40 mL, twice), and with brine. The organic layer was dried over sodium sulphate and the solvent was removed under vacuum to give a residue (275 mg) that was used for the next step without further purification.

Yield: 85% (Pale yellow oil); [α]_{D}²⁰=−8.7 (c=0.615, CHCl₃).

¹H-NMR (CDCl₃, 300 MHz), δ (ppm): 7.43 (dd, 2H); 7.08 (dd, 2H); 3.99-3.19 (m br, 5H); 1.98-1.42 (m br, 4H).

### 14 (B) [(S)-3-(5-Benzyl-tetrazol-2-yl)-piperidin-1-yl]-(4-fluoro-phenyl)-methanone

Diisopropylazadicarboxylate (DIAD, 141 uL, 0.7 mmol) was added to a cooled mixture of benzyltetrazole (112 mg, 0.7 mmol), (4-fluoro-phenyl)-((R)-3-hydroxy-piperidin-1-yl)-methanone (100 mg, 0.36 mmol) and solid supported triphenylphosphine (PS-PPh₃, ex Argonaut Technologies, loading 2.4 mmol/g, 420 mg, 1.0 mmol) in DCM (4 mL), at 0° C. The mixture was then heated under microwave irradiation for 30 min at 100° C.

The resin was filtered off, washed with dichloromethane and the filtrate was evaporated under reduced pressure. The residue was first purified by flash chromatography (silica gel, eluent gradient: from DCM to DCM/MeOH 98:2). The crude material thus recovered was then dissolved in toluene and passed through a silica gel cartridge (Isolute Flash II 2 g, eluent gradient: starting with hexane, then with hexane/diethyl ether 75:25, then with hexane/diethyl ether 6:4, then with DCM/MeOH 98:2).

The title compound was obtained pure as a colourless gum (32 mg).

Yield: 25%; (colourless gum); LCMS (RT): 6.69 min (Method: C); MS (ES+) gave m/z: 366.2 (MH+).

¹H-NMR (DMSO-d₆ 368K), δ (ppm): 7.39 (dd, 2H); 7.34-7.21 (m, 5H); 7.18 (dd, 2H); 4.97 (m, 1H); 4.23 (s, 2H); 4.23 (m, 1H); 3.75 (dd, 1H); 3.66 (ddd, 1H); 3.41 (ddd, 1H); 2.36 (m, 1H); 2.26 (m, 1H); 1.90 (m, 1H); 1.70 (m, 1H).

### Example 15

### {3-[3-(4-Fluoro-phenoxy)-[1,2,4]oxadiazol-5-yl]-piperidin-1-yl}-(4-fluoro-phenyl)-methanone

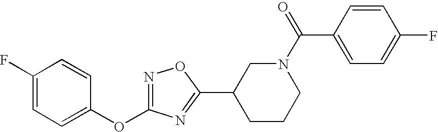

### 15 (A) [3-(3-Bromo-[1,2,4]oxadiazol-5-yl)-piperidin-1-yl]-(4-fluoro-phenyl)-methanone

To a stirred solution of glyoxylic acid (5 g, 54.3 mmol) in water (40 mL), hydroxylamine hydrochloride (4.9 g, 70.6 mmol) was added and the mixture was stirred for 24 h at room temperature. The reaction mixture was then diluted with DCM (50 mL) and NaHCO₃ (9 g, 102 mmol) was carefully added in portions. While cooling at 0° C., a solution of bromine (56 mL) in 25 mL of DCM was slowly added and stirring at 0° C. was maintained for 3 h. The phases were separated, the organic layer was washed with water, dried over sodium sulphate and evaporated under reduced pressure to give 2 g of dibromoformaldoxime. Dibromoformaldoxime (154 mg, 0.76 mmol) was added portionwise over 45 minutes to a heated solution of 1-(4-Fluoro-benzoyl)-piperidine-3-carbonitrile (320 mg, 1.52 mmol), prepared as described in Example 5 (A), and NaHCO₃ (204 mg, 2.4 mmol) in toluene at 90° C. After stirring for 2 h, another 154 mg of dibromoformaldoxime were added and heating at 90° C. was kept for 6 h. Another 300 mg of dibromoformaldoxime and 500 mg of NaHCO₃ were added in small portions and stirring at 90° C. was maintained for 10 h. The solution mixture was cooled and diluted with water and ethyl acetate, the phases were separated. The organic layer was washed with water, dried over sodium sulphate and evaporated under reduced pressure to give a crude residue that was purified by flash chromatography (silica gel, eluent: petroleum ether/ethyl acetate 1:1). [3-(3-Bromo-[1,2,4]oxadiazol-5-yl)-piperidin-1-yl]-(4-fluoro-phenyl)-methanone was obtained as a solid (152 mg).

Yield: 28%; LCMS (RT): 6.82 min (Method B); MS (ES+) gave m/z: 354.0.

¹H-NMR (DMSO-d₆, 343K), δ (ppm): 7.45 (dd, 2H); 7.24 (dd, 2H); 4.15 (m, 1H); 3.73 (m, 1H); 3.51 (dd, 1H); 3.40 (ddd, 1H); 3.27 (ddd, 1H); 2.20 (m, 1H); 1.92 (m, 1H); 1.77 (m, 1H); 1.63 (m, 1H).

### 15 (B) {3-[3-(4-Fluoro-phenoxy)-[1,2,4]oxadiazol-5-yl]-piperidin-1-yl}-(4-fluoro-phenyl)-methanone

A mixture of [3-(3-bromo-[1,2,4]oxadiazol-5-yl)-piperidin-1-yl]-(4-fluoro-phenyl)methanone (152 mg, 0.43 mmol), 4-fluorophenol (193 mg, 1.72 mmol), K₂CO₃ (402 mg, 1.72 mmol) in dioxane (5 mL) was refluxed overnight. 50 mL of Na₂CO₃ (aq) and ethyl acetate were added, the phases were separated, the organic layer was dried over sodium sulphate and evaporated under reduced pressure to give a crude residue that was purified by flash chromatography (silica gel, eluent: petroleum ether/ethyl acetate 6:4). {3-[3-(4-Fluoro-phenoxy)-[1,2,4]oxadiazol-5-yl]-piperidin-1-yl}-(4-fluoro-phenyl)-methanone was obtained as a white solid (50 mg).

Yield: 30% (white solid); LCMS (RT): 6.99 min (Method C); MS (ES+) gave m/z: 386.2 (MH+).

¹H-NMR (DMSO-d₆, 343K), δ (ppm): 7.46-7.35 (m, 4H); 7.30-7.18 (m, 4H); 4.13 (m, 1H); 3.71 (m, 1H); 3.49 (dd, 1H); 3.36-3.21 (m, 2H); 2.17 (m, 1H); 1.91 (m, 1H); 1.75 (m, 1H); 1.61 (m, 1H).

### Example 16

### (4-Fluoro-phenyl)-[3-(3-phenoxy-[1,2,4]oxadiazol-5-yl)-piperidin-1-yl]-methanone

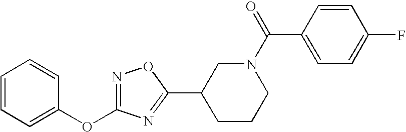

The title compound was obtained following the same procedure described in Example 15(B), starting from [3-(3-Bromo-[1,2,4]oxadiazol-5-yl)-piperidin-1-yl]-(4-fluoro-phenyl)-methanone (prepared as described in Example 15(A)) and phenol. Purification by flash chromatography (silica gel, eluent: petroleum ether/ethyl acetate 7:3) gave 44 mg of (4-Fluoro-phenyl)-[3-(3-phenoxy-[1,2,4]oxadiazol-5-yl)-piperidin-1-yl]-methanone.

Yield: 27% (off-white solid); mp=96-100° C.; LCMS (RT): 7.32 min (Method C); MS (ES+) gave m/z: 368.1 (MH+).

¹H-NMR (DMSO-d₆, 343K), δ (ppm): 7.49-7.39 (m, 4H); 7.34-7.18 (m, 5H); 4.14 (m, 1H); 3.71 (m, 1H); 3.50 (dd, 1H); 3.37-3.20 (m, 2H); 2.18 (m, 1H); 1.90 (m, 1H); 1.76 (m, 1H); 1.60 (m, 1H).

### Example 17

### (6-Fluoro-pyridin-3-yl)-[(S)-3-(3-phenoxy-[1,2,4]oxadiazol-5-yl)-piperidin-1-yl]-methanone

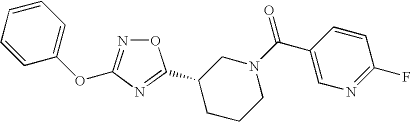

### 17 (A) (S)-3-(3-Bromo-[1,2,4]oxadiazol-5-yl)-piperidine-1-carboxylic acid tert-butyl ester

(S)-3-(3-Bromo-[1,2,4]oxadiazol-5-yl)-piperidine-1-carboxylic acid tert-butyl ester was prepared following the experimental procedure described in Example 15 (A), starting from (S)-3-cyano-piperidine-1-carboxylic acid tert-butyl ester, prepared as described in Example 3(B), and dibromoformaldoxime. Purification by flash chromatography (silica gel, eluent gradient: from petroleum ether to petroleum ether/ethyl acetate 1:1) afforded the pure desired product.

Yield: 25%; LCMS (RT): 5.84 min (Method E); MS (ES+) gave m/z: 332.1 and 334.1.

### 17 (B) (S)-3-(3-Phenoxy-[1,2,4]oxadiazol-5-yl)-piperidine hydrochloride

A mixture of (S)-3-(3-bromo-[1,2,4]oxadiazol-5-yl)-piperidine-1-carboxylic acid tert-butyl ester (250 mg, 0.75 mmol), phenol (105 mg, 1.12 mmol), Cs₂CO₃ (489 mg, 1.5 mmol) in dioxane (5 mL) was heated at 90° C. overnight. The solvent was removed under reduced pressure and the residue was partitioned between ethyl acetate and Na₂CO₃ (aq). The phases were separated, the organic layer was dried over sodium sulphate and evaporated under reduced pressure to afford (S)-3-(3-phenoxy-[1,2,4]oxadiazol-5-yl)-piperidine-1-carboxylic acid tert-butyl ester, which was used for the next step without further purification.

To a solution of (S)-3-(3-phenoxy-[1,2,4]oxadiazol-5-yl)-piperidine-1-carboxylic acid tert-butyl ester (0.75 mmol) in DCM (2 mL), cooled at 0° C., 4N HCl (dioxane solution, 1 mL) was added dropwise. After stirring at RT for 2 h, the solvent was removed and the crude was purified by passing it through a SCX cartridge (eluent: starting with methanol then with 5% NH₃ in MeOH). 60 mg of the pure title compound were obtained.

Yield: 33% (white solid); LCMS (RT): 2.8 min (Method E); MS (ES+) gave m/z: 246.3.

### 17 (C) (6-Fluoro-pyridin-3-yl)-[(S)-3-(3-phenoxy-[1,2,4]oxadiazol-5-yl)-piperidin-1-yl]-methanone

A mixture of (S)-3-(3-phenoxy-[1,2,4]oxadiazol-5-yl)-piperidine hydrochloride (60 mg, 0.24 mmol), 6-fluoronicotinic acid (41 mg, 0.29 mmol), HOAT (39 mg, 0.29 mmol), EDCI.HCl (69 mg, 0.36 mmol) and dry triethylamine (67 uL, 0.48 mmol) in DCM (5 mL) was kept under stirring overnight at ambient temperature, under nitrogen atmosphere. The residue was diluted with water (40 mL), the phases were separated and the organic layer was washed with Na₂CO₃ 1N (40 mL, twice) and with brine. The organic layer was dried over sodium sulphate and the solvent was removed under vacuum to give a residue that was purified by flash chromatography (silica gel, eluent: petroleum ether/ethyl acetate 6:4) to give the pure title compound (30 mg).

Yield: 34% (Colourless oil); LCMS (RT): 2.74 min (Method F); MS (ES+) gave m/z: 369.1 (MH+).

¹H-NMR (DMSO-d₆, 353K), δ (ppm): 8.28 (m, 1); 7.98 (ddd, 1H); 7.46 (dd, 2H); 7.34-7.25 (m, 3H); 7.19 (ddd, 1H); 4.14 (m, 1H); 3.71 (m, 1H); 3.54 (dd, 1H); 3.34 (m, 2H); 2.19 (m, 1H); 1.94 (m, 1H); 1.77 (m, 1H); 1.65 (m, 1H).

### Example 18

### {(S)-3-[3-(2-Fluoro-phenoxy)-[1,2,4]oxadiazol-5-yl]-piperidin-1-yl}-(6-fluoro-pyridin-3-yl)-methanone

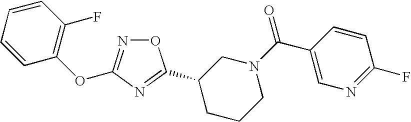

### 18 (A) (S)-3-[3-(2-Fluoro-phenoxy)-[1,2,4]oxadiazol-5-yl]-piperidine hydrochloride

The title compound was obtained following the same procedure described in Example 17(B), starting from (S)-3-(3-bromo-[1,2,4]oxadiazol-5-yl)-piperidine-1-carboxylic acid tert-butyl ester (prepared as described in Example 17(A)) and 2-fluorophenol.

Yield: 33% (white solid).

### 18 (B) {(S)-3-[3-(2-Fluoro-phenoxy)-[1,2,4]oxadiazol-5-yl]-piperidin-1-yl}-(6-fluoro-pyridin-3-yl)-methanone

The title compound was obtained following the same procedure described in Example 17(C), starting from (S)-3-[3-(2-Fluoro-phenoxy)-[1,2,4]oxadiazol-5-yl]-piperidine hydrochloride and 6-fluoronicotinic acid. Purification by flash chromatography (silica gel, eluent: petroleum ether/ethyl acetate 7:3) gave 40 mg of {(S)-3-[3-(2-Fluoro-phenoxy)-[1,2,4]oxadiazol-5-yl]-piperidin-1-yl}-(6-fluoro-pyridin-3-yl)-methanone.

Yield: 14% (yellow oil); LCMS (RT): 2.77 min (Method F); MS (ES+) gave m/z: 387.1 (MH+).

¹H-NMR (DMSO-d₆, 353K), δ (ppm): 8.27 (d, 1H); 7.97 (ddd, 1H); 7.49 (ddd, 1H); 7.43-7.23 (m, 3H); 7.19 (dd, 1H); 4.13 (m, 1H); 3.70 (m, 1H); 3.54 (dd, 1H); 3.41-3.28 (m, 2H); 2.18 (m, 1H); 1.93 (m, 1H); 1.77 (m, 1H); 1.65 (m, 1H).

### Example 19

### {(S)-3-[3-(3-Fluoro-phenoxy)-[1,2,4]oxadiazol-5-yl]-piperidin-1-yl}-(6-fluoro-pyridin-3-yl)-methanone

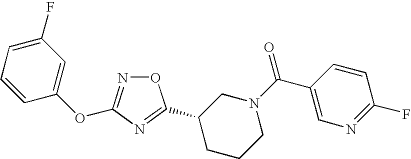

### 19 (A) (S)-3-[3-(3-Fluoro-phenoxy)-[1,2,4]oxadiazol-5-yl]-piperidine hydrochloride

The title compound was obtained following the same procedure described in Example 17(B), starting from (S)-3-(3-bromo-[1,2,4]oxadiazol-5-yl)-piperidine-1-carboxylic acid tert-butyl ester (prepared as described in Example 17(A)) and 3-fluorophenol.

### 19 (B) {(S)-3-[3-(3-Fluoro-phenoxy)-[1,2,4]oxadiazol-5-yl]-piperidin-1-yl}-(6-fluoro-pyridin-3-yl)-methanone

The title compound was obtained following the same procedure described in Example 17(C), starting from (S)-3-[3-(3-fluoro-phenoxy)-[1,2,4]oxadiazol-5-yl]-piperidine hydrochloride and 6-fluoronicotinic acid. Purification by flash chromatography (silica gel, eluent: petroleum ether/ethyl acetate 7:3) gave 45 mg of {(S)-3-[3-(3-fluoro-phenoxy)-[1,2,4]oxadiazol-5-yl]-piperidin-1-yl}-(6-fluoro-pyridin-3-yl)-methanone.

Yield: 14% (yellow oil); LCMS (RT): 2.84 min (Method F); MS (ES+) gave m/z: 387.1 (MH+).

¹H-NMR (DMSO-d₆, 353K), δ (ppm): 8.28 (m, 1H); 7.99 (ddd, 1H); 7.49 (m, 1H); 7.26-7.16 (m, 3H); 7.12 (ddd, 1H); 4.14 (m, 1H); 3.71 (m, 1H); 3.55 (dd, 1H); 3.41-3.28 (m, 2H); 2.19 (m, 1H); 1.94 (m, 1H); 1.77 (m, 1H); 1.65 (m, 1H).

### Example 20

### (4-Fluoro-phenyl)-[(S)-3-(3-phenylsulfanyl-[1,2,4]oxadiazol-5-yl)-piperidin-1-yl]-methanone

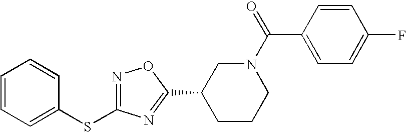

### 20 (A) [(S)-3-(3-Bromo-[1,2,4]oxadiazol-5-yl)-piperidin-1-yl]-(4-fluoro-phenyl)-methanone

The title compound was obtained following the same procedure described in Example 15(A), starting from (S)-1-(4-Fluoro-benzoyl)-piperidine-3-carbonitrile, prepared as described in Example 5(A).

Yield: 22%.

¹H-NMR (DMSO-d₆ 300 MHz, 343K), δ (ppm): 7.45 (dd, 2H); 7.24 (dd, 2H); 4.15 (m, 1H); 3.73 (m, 1H); 3.51 (dd, 1H); 3.40 (ddd, 1H); 3.27 (ddd, 1H); 2.20 (m, 1H); 1.92 (m, 1H); 1.77 (m, 1H); 1.63 (m, 1H).

### 20 (B) (4-Fluoro-phenyl)-[(S)-3-(3-phenylsulfanyl-[1,2,4]oxadiazol-5-yl)-piperidin-1-yl]-methanone

The title compound was obtained following the same procedure described in Example 15(B), starting from [(S)-3-(3-bromo-[1,2,4]oxadiazol-5-yl)-piperidin-1-yl]-(4-fluoro-phenyl)-methanone and thiophenol.

Purification by flash chromatography (silica gel, eluent: petroleum ether/ethyl acetate 1:1) and subsequent preparative HPLC gave (4-fluoro-phenyl)-[(S)-3-(3-phenylsulfanyl-[1,2,4]oxadiazol-5-yl)-piperidin-1-yl]-methanone as a colourless oil (6 mg).

Yield: 2% (colourless oil); LCMS (RT): 7.61 min (Method C); MS (ES+) gave m/z: 384.1 (MH+).

¹H-NMR (DMSO-d₆, 343K), δ (ppm): 7.62 (m, 2H); 7.48-7.38 (m, 5H); 7.21 (dd, 2H); 4.11 (m, 1H), 3.69 (m, 1H); 3.48 (dd, 1H); 3.33 (ddd, 1H); 3.26 (ddd, 1H); 2.16 (m, 1H); 1.89 (m, 1H); 1.75 (m, 1H); 1.60 (m, 1H).

### Example 21

### {3-[3-(3-Fluoro-phenoxy)-[1,2,4]oxadiazol-5-yl]-piperidin-1-yl}-(4-fluoro-phenyl)-methanone

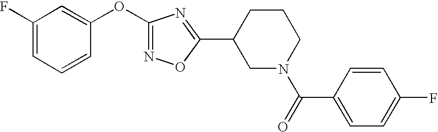

A mixture of [3-(3-bromo-[1,2,4]oxadiazol-5-yl)-piperidin-1-yl]-(4-fluoro-phenyl)-methanone (210 mg, 0.59 mmol), prepared as described in Example 15 (A), 3-fluorophenol (81 uL, 0.89 mmol), Cs₂CO₃ (386 mg, 1.18 mmol) in dioxane (5 mL) was heated at 90° C. for 8 h. The solvent was evaporated off and 50 mL of Na₂CO₃ (aq) and ethyl acetate were added, the phases were separated, the organic layer was dried over sodium sulphate and evaporated under reduced pressure to give a crude residue that was purified by flash chromatography (silica gel, eluent gradient: from petroleum ether to petroleum ether/ethyl acetate 1:1). {{3-[3-(3-Fluoro-phenoxy)-[1,2,4]oxadiazol-5-yl]-piperidin-1-yl}-(4-fluoro-phenyl)-methanone was obtained as a pale yellow solid (43 mg).

Yield: 19% (pale yellow solid); mp=99-102° C.; LCMS (RT): 2.49 min (Method G);

MS (ES+) gave m/z: 386.1 (MH+).

¹H-NMR (DMSO-d₆, 353K), δ (ppm): 7.54-7.40 (m, 3H); 7.27-7.09 (m, 5H); 4.14 (m, 1H); 3.71 (m, 1H); 3.5 1 (dd, 1H); 3.38-3.23 (m, 2H); 2.19 (m, 1H); 1.93 (m, 1H); 1.78 (m, 1H); 1.61 (m, 1H).

### Example 22

### (3-Fluoro-pyridin-4-yl)-[(S)-3-(3-phenoxy-[1,2,4]oxadiazol-5-yl)-piperidin-1-yl]-methanone

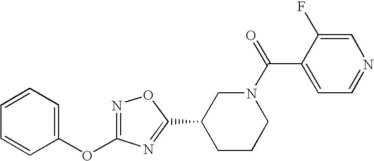

### 22 (A) (S)-3-(3-Phenoxy-[1,2,4]oxadiazol-5-yl)-piperidine-1-carboxylic acid tert-butyl ester

A solution of cyanato-benzene (300 mg, 2.52 mmol), prepared as described in J. Am. Chem. Soc.; 2005, 2408-2409, in diethyl ether (20 mL) was dropped over 15 minutes into a solution of O-THP protected hydroxylamine (900 mg, 7.56 mmol) cooled at 0° C. 20 mL of THF were added. The solution was stirred at RT overnight, solvent was removed and the crude yellow oil obtained was used for the next step without further purification.

The yellow oil was dissolved in THF (30 mL) and 4N HCl (dioxane solution, 2 mL) was added at 0° C. The mixture was stirred at room temperature for 15 h, solvent was removed and 1.25 g of pale yellow solid were obtained. The solid was dissolved in dioxane (25 mL) and added to a mixture of (S)—N-Boc-nipecotic acid (580 mg, 2.52 mmol), HOBT (340 mg, 2.52 mmol), EDCI.HCl (725 mg, 3.78 mmol) and dry triethylamine (350 uL, 2.52 mmol) in dioxane (25 mL). The mixture was kept under stirring for 15 h at 80° C., under nitrogen atmosphere. The solvent was removed and the residue was diluted with DCM (40 mL) and washed with Na₂CO₃ 1N (40 mL, twice) and with brine. The organic layer was dried over sodium sulphate and the solvent was removed under vacuum to give a dark brown oil that was in turn dissolved in acetonitrile (5 mL), few activated 4A molecular sieves were added and the mixture was heated at 100° C. for 2 h, in a sealed tube, in a microwaves oven. Solvent was removed, the resulting brown oil was purified by flash chromatography (silica gel, eluent:hexane/ethyl acetate 8:2). 100 mg of (S)-3-(3-phenoxy-[1,2,4]oxadiazol-5-yl)-piperidine-1-carboxylic acid tert-butyl ester were obtained.

Yield: 12% (yellow oil); LCMS (RT): 3.76 min (Method L); MS (ES+) gave m/z: 346.09.

### 22 (B) (S)-3-(3-Phenoxy-[1,2,4]oxadiazol-5-yl)-piperidine hydrochloride

(S)-3-(3-Phenoxy-[1,2,4]oxadiazol-5-yl)-piperidine-1-carboxylic acid tert-butyl ester (0.07 g, 0.203 mmol) was dissolved in dioxane (1 mL) and 1 mL of HCl 4N (dioxane solution) was added dropwise at 0° C. The resulting mixture was stirred at room temperature for 3 h. The solvent was evaporated under reduced pressure to afford 55 mg (yield: 96%) of (S)-3-(3-phenoxy-[1,2,4]oxadiazol-5-yl)-piperidine hydrochloride as a yellow gummy solid.

LCMS (RT): 2.53 min (Method L); MS (ES+) gave m/z: 246.1.

### 22 (C) (3-Fluoro-pyridin-4-yl)-[(S)-3-(3-phenoxy-[1,2,4]oxadiazol-5-yl)-piperidin-1-yl]-methanone

A mixture of (S)-3-(3-phenoxy-[1,2,4]oxadiazol-5-yl)-piperidine hydrochloride (55 mg, 0.195 mmol), 3-fluoro-pyridine-4-carboxylic acid (28 mg, 0.195 mmol), HOAT (26 mg, 0.195 mmol), EDCI.HCl (56 mg, 0.293 mmol) and dry triethylamine (82 uL, 0.586 mmol) in DCM (5 mL) was kept under stirring for 15 h at ambient temperature, under nitrogen atmosphere. Solvent was removed. The residue was purified by flash chromatography (silica gel, eluent: hexane/ethyl acetate 1:9) to give the pure title compound (50 mg).

Yield: 70% (Pale yellow gummy solid); [α]_{D}²⁰=+77.3 (c=0.76, MeOH); LCMS (RT): 2.05 min (Method H); MS (ES+) gave m/z: 369.2 (MH+).

¹H-NMR (DMSO-d₆, 373K), δ (ppm): 8.61 (s br 1H); 8.50 (dd 1H); 7.46 (m 2H); 7.39 (dd 1H); 7.34-7.26 (m 3H); 4.12 (m br 2H); 3.58 (dd 1H); 3.31 (m 2H); 2.21 (m 1H); 1.97 (m 1H); 1.80 (m 1H); 1.63 (m 1H).

Compounds in Table 1 were prepared following the procedures described in Example 22 (C), starting from (S)-3-(3-phenoxy-[1,2,4]oxadiazol-5-yl)-piperidine hydrochloride, prepared as described in Example 22 (B), and the corresponding commercially available carboxylic acids.

TABLE 1

| Ex. | Name | Analytical details |
|---|---|---|
| 23 | (4-Methylphenyl)-[(S)-3-(3- | LCMS (RT): 5.68 min (Method E); MS (ES+) |
| | phenoxy-[1,2,4]oxadiazol-5- | gave m/z: 364.1 (MH+) |
| | yl)-piperidin-1-yl]-methanone | [α]_{D}²⁰ = +54.9 (c = 0.75, MeOH) |
| 24 | (2-Methoxy-phenyl)-[(S)-3- | LCMS (RT): 2.42 min (Method G); MS (ES+) |
| | (3-phenoxy- | gave m/z: 380.1 (MH+) |
| | [1,2,4]oxadiazol-5-yl)- | ¹H-NMR (DMSO-d₆, 353K), δ (ppm): 7.46 (m, |
| | piperidin-1-yl]-methanone | 2H); 7.38-7.25 (m, 4H); 7.01 (ddd, 1H); 6.92 (m, |
| | | 2H); 4.15 (m, 1H); 3.80 (s, 3H); 3.73 (m, 1H); |
| | | 3.48 (dd, 1H); 3.36-3.21 (m, 2H); 2.19 (m, 1H); |
| | | 1.92 (m, 1H); 1.78 (m, 1H); 1.61 (m, 1H) |
| | | [α]_{D}²⁰ = +77.5 (c = 1.03, MeOH) |
| 25 | [(S)-3-(3-Phenoxy- | LCMS (RT): 4.5 min (Method E); MS (ES+) |
| | [1,2,4]oxadiazol-5-yl)- | gave m/z: 351.1 (MH+) |
| | piperidin-1-yl]-pyridin-2-yl-methanone | [α]_{D}²⁰ = +77.2 (c = 0.83, MeOH) |
| 26 | (2-Fluoro-pyridin-4-yl)-[(S)- | LCMS (RT): 4.95 min (Method E); MS (ES+) |
| | 3-(3-phenoxy-[1,2,4]oxadiazol-5-yl)- | gave m/z: 369.1 (MH+) |
| | piperidin-1-yl]-methanone | [α]_{D}²⁰ = +69.2 (c = 1.05, MeOH) |
| 27 | (3H-Imidazol-4-yl-[(S)-3- | LCMS (RT): 3.24 min (Method E); MS (ES+) |
| | (3-phenoxy- | gave m/z: 340.1 (MH+) |
| | [1,2,4]oxadiazol-5-yl)- | |
| | piperidin-1-yl]-methanone | |
| 28 | (3,5-Difluoro-phenyl)-[(S)- | LCMS (RT): 5.66 min (Method E); MS (ES+) |
| | 3-(3-phenoxy- | gave m/z: 386.1 (MH+) |
| | [1,2,4]oxadiazol-5-yl)- | [α]_{D}²⁰ = +56.1 (c = 0.82, MeOH) |
| | piperidin-1-yl]-methanone | |
| 29 | (5-Methyl-isoxazol-4-yl)- | LCMS (RT): 4.88 min (Method E); MS (ES+) |
| | [(S)-3-(3-phenoxy- | gave m/z: 355.1 (MH+) |
| | [1,2,4]oxadiazol-5-yl)- | [α]_{D}²⁰ = +82.5 (c = 0.45, MeOH) |
| | piperidin-1-yl]-methanone | |
| 30 | [(S)-3-(3-Phenoxy- | LCMS (RT): 4.54 min (Method E); MS (ES+) |
| | [1,2,4]oxadiazol-5-yl)- | gave m/z: 357.1 (MH+) |
| | piperidin-1-yl]-thiazol-5-yl-methanone | |

Compounds in Table 2 were prepared following the procedures described in Example 1 (C), starting from (S)-3-(3-phenoxy-[1,2,4]oxadiazol-5-yl)-piperidine hydrochloride, prepared as described in Example 22 (B), and the corresponding commercially available acyl chlorides.

TABLE 2

| Ex. | Name | Analytical details |
|---|---|---|
| 31 | [(S)-3-(3-Phenoxy- | LCMS (RT): 2.38 min (Method G); MS (ES+) |
| | [1,2,4]oxadiazol-5-yl)- | gave m/z: 350.1 (MH+) |
| | piperidin-1-yl]-phenyl- | ¹H-NMR (DMSO-d₆, 353K), δ (ppm): |
| | methanone | 7.50-7.26 (m, 10H); 4.16 (m, 1H); 3.73 (m, 1H); 3.49 (dd, |
| | | 1H); 3.35-3.21 (m, 2H); 2.19 (m, 1H); 1.92 (m, |
| | | 1H); 1.79 (m, 1H); 1.60 (m, 1H) |
| | | [α]_{D}²⁰ = +82.8 (c = 1.07, MeOH) |
| 32 | (4-Chloro-phenyl)-[(S)-3-(3- | LCMS (RT): 2.58 min (Method G); MS (ES+) |
| | phenoxy-[1,2,4]oxadiazol-5- | gave m/z: 384.1 (MH+) |
| | yl)-piperidin-1-yl]- | ¹H-NMR (DMSO-d₆, 353K), δ (ppm): |
| | methanone | 7.52-7.26 (m, 9H); 4.13 (m, 1H); 3.70 (m, 1H); 3.50 (dd, |
| | | 1H); 3.36-3.23 (m, 2H); 2.18 (m, 1H); 1.92 (m, |
| | | 1H); 1.76 (m, 1H); 1.62 (m, 1H) |
| | | [α]_{D}²⁰ = +87.7 (c = 1.01, MeOH) |
| 33 | (4-Methoxy-phenyl)-[(S)-3- | LCMS (RT): 2.39 min (Method G); MS (ES+) |
| | (3-phenoxy- | gave m/z: 380.0 (MH+) |
| | [1,2,4]oxadiazol-5-yl)- | ¹H-NMR (DMSO-d₆, 353K), δ (ppm): 7.46 (dd, |
| | piperidin-1-yl]-methanone | 2H); 7.37-7.26 (m, 5H); 6.97 (d, 1H); 4.18 (dd |
| | | br, 1H); 3.82 (s, 3H); 3.77 (m, 1H); 3.48 (dd, |
| | | 1H); 3.34-3.21 (m, 2H); 2.19 (m, 1H); 1.91 (m, |
| | | 1H); 1.77 (m, 1H); 1.61 (m, 1H) |
| | | [α]_{D}²⁰ = +88.5 (c = 1.05, MeOH) |
| 34 | (3,4-Dichloro-phenyl)-[(S)- | LCMS (RT): 2.75 min (Method G); MS (ES+) |
| | 3-(3-phenoxy- | gave m/z: 418.1 (MH+) |
| | [1,2,4]oxadiazol-5-yl)- | ¹H-NMR (DMSO-d₆, 353K), δ (ppm): 7.67 (d, |
| | piperidin-1-yl]-methanone | 1H); 7.61 (d, 1H); 7.46 (dd, 2H); 7.38-7.25 (m, |
| | | 4H); 4.11 (m, 1H); 3.68 (m, 1H); 3.51 (dd, 1H); |
| | | 3.39-3.25 (m, 2H); 2.18 (m, 1H); 1.92 (m, 1H); |
| | | 1.76 (m, 1H); 1.63 (m, 1H) |
| | | [α]_{D}²⁰ = +70.1 (c = 0.96, MeOH) |
| 35 | (3-Methoxy-phenyl)-[(S)-3- | LCMS (RT): 5.41 min (Method E); MS (ES+) |
| | (3-phenoxy- | gave m/z: 380.1 (MH+) |
| | [1,2,4]oxadiazol-5-yl)- | ¹H-NMR (DMSO-d₆ 373K), δ (ppm): |
| | piperidin-1-yl]-methanone | 7.49-7.25 (m, 6H); 7.15 (dd, 1H); 7.07 (dd, 1H); |
| | | 6.99 (ddd, 1H); 4.44-3.53 (m br, 2H); 3.82 (s, 3H); |
| | | 3.45 (m, 1H); 3.29-3.09 (m, 2H); 2.19 (m, 1H); |
| | | 1.92 (m, 1H); 1.77 (m, 1H); 1.57 (m, 1H) |
| | | [α]_{D}²⁰ = +71.6 (c = 1.01, MeOH) |
| 36 | (2-Methyl-phenyl)-[(S)-3- | LCMS (RT): 5.60 min (Method E); MS (ES+) |
| | (3-phenoxy- | gave m/z: 364.1 (MH+) |
| | [1,2,4]oxadiazol-5-yl)- | [α]_{D}²⁰ = +64.0 (c = 0.90, MeOH) |
| | piperidin-1-yl]-methanone | |
| 37 | (2-Fluoro-phenyl)-[(S)-3-(3- | LCMS (RT): 5.4 min (Method E); MS (ES+) |
| | phenoxy-[1,2,4]oxadiazol-5- | gave m/z: 368.1 (MH+) |
| | yl)-piperidin-1-yl]- | [α]_{D}²⁰ = +76.9 (c = 0.91, MeOH) |
| | methanone | |
| 38 | (3-Fluoro-phenyl)-[(S)-3-(3- | LCMS (RT): 5.51 min (Method E); MS (ES+) |
| | phenoxy-[1,2,4]oxadiazol-5- | gave m/z: 368.1 (MH+) |
| | yl)-piperidin-1-yl]- | [α]_{D}²⁰ = +73.9 (c = 0.85, MeOH) |
| | methanone | |
| 39 | [(S)-3-(3-Phenoxy- | LCMS (RT): 3.44 min (Method E); MS (ES+) |
| | [1,2,4]oxadiazol-5-yl)- | gave m/z: 351.1 (MH+) |
| | piperidin-1-yl]-pyridin-3-yl- | |
| | methanone | |
| 40 | [(S)-3-(3-Phenoxy- | LCMS (RT): 3.64 min (Method E); MS (ES+) |
| | [1,2,4]oxadiazol-5-yl)- | gave m/z: 351.1 (MH+) |
| | piperidin-1-yl]-pyridin-4-yl- | [α]_{D}²⁰ = +81.4 (c = 0.85, MeOH) |
| | methanone | |
| 41 | (3,5-Dimethyl-isoxazol-4- | LCMS (RT): 4.90 min (Method E); MS (ES+) |
| | yl)-[(S)-3-(3-phenoxy- | gave m/z: 369.1 (MH+) |
| | [1,2,4]oxadiazol-5-yl)- | |
| | piperidin-1-yl]-methanone | |
| 42 | (4-Fluoro-phenyl)-[(S)-3-(3- | LCMS (RT): 2.43 min (Method G); MS (ES+) |
| | phenoxy-[1,2,4]oxadiazol-5- | gave m/z: 368.1 (MH+) |
| | yl)-piperidin-1-yl]- | ¹H-NMR (DMSO-d₆, 353K), δ (ppm): 7.45 (m, |
| | methanone | 4H); 7.31 (m, 3H); 7.23 (dd, 2H); 4.14 (m, 1H); |
| | | 3.71 (m, 1H); 3.50 (dd, 1H); 3.36-3.22 (m, 2H); |
| | | 2.19 (m, 1H); 1.92 (m, 1H); 1.76 (m, 1H); |
| | | 1.60 (m, 1H) |
| | | [α]_{D}²⁰ = +86.9 (c = 1.01, MeOH) |

### Example 43

### {(S)-3-[3-(3-Fluoro-phenoxy)-[1,2,4]oxadiazol-5-yl]-piperidin-1-yl}-(4-fluoro-phenyl)-methanone

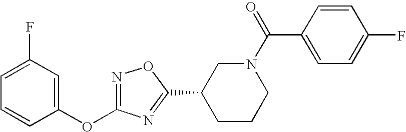

### 43 (A) (S)-3-[3-(3-Fluoro-phenoxy)-[1,2,4]oxadiazol-5-yl]-piperidine-1-carboxylic acid tert-butyl ester

A solution of cyanato-3-fluorobenzene (6.2 g, 45 mmol), prepared as described in J. Am. Chem. Soc.; 2005, 2408-2409, in THF (10 mL) was dropped over 15 minutes into a solution of O-THP protected hydroxylamine (7.5 g, 64.35 mmol) in THF (50 mL) cooled at 0° C. The solution was stirred at RT overnight Solvent was removed and the crude brown oil (13.6 g) was used for the next step without further purification.

The residue was dissolved in THF (60 mL) and 4N HCl (dioxane solution, 22.5 mL) was added at 0° C. The mixture was stirred at room temperature for 24 h. The solvent was removed and beige gummy solid was obtained. It was dissolved in dioxane (320 mL) and HOBT (6.9 g, 45 mmol), EDCI.HCl (8.6 g, 45 mmol) were added. The reaction was heated at 50° C. for 2 h. (S)—N-Boc-nipecotic acid (6.9 g, 45 mmol) and dry triethylamine (6.3 mL, 45 mmol) were added and the reaction mixture was heated at 80° C. for 2 h. The solvent was removed and the residue was diluted with DCM and washed with 1M Na₂CO₃ and with brine. The organic layer was dried over sodium sulphate and the solvent was evaporated under vacuum to give a dark brown oil (24 g). The oil was dissolved in toluene (150 mL) and the mixture was refluxed for 20 h removing water with Dean-Stark apparatus. The solvent was evaporated and the resulting oil was dissolved in Et₂O and washed with 0.5N NaOH. The resulting brown oil was purified by flash chromatography (silica gel, eluent:Petroleum ether/ethyl acetate 6:1) to yield 7.1 g of the title compound as a viscous oil.

Yield: 43.5%; LCMS (RT): 1.79 min (Method M); MS (ES+) gave m/z: 364 (MH+) [α]_{D}²⁰=+55.7 (c=1.12, MeOH)

### 43 (B) (S)-3-[3-(3-Fluoro-phenoxy)-[1,2,4]oxadiazol-5-yl]-piperidine hydrochloride

(S)-3-[3-(3-Fluoro-phenoxy)-[1,2,4]oxadiazol-5-yl]-piperidine-1-carboxylic acid tert-butyl ester (7.1 g, 19.56 mmol) was dissolved in DCM (150 mL) and 39.1 mL of 4N HCl (dioxane solution) was added dropwise at 0° C. The resulting mixture was stirred at room temperature for 20 h. The solvent was evaporated under reduced pressure to afford 5.85 g of (S)-3-[3-(3-fluoro-phenoxy)-[1,2,4]oxadiazol-5-yl]-piperidine hydrochloride as a yellow gummy solid.

LCMS (RT): 1.01 min (Method M); MS (ES+) gave m/z: 264.0 (MH+)

### 43 (C) {(S)-3-[3-(3-Fluoro-phenoxy)-[1,2,4]oxadiazol-5-yl]-piperidin-1-yl}-(4-fluoro-phenyl)-methanone

To a suspension of (S)-3-[3-(3-fluoro-phenoxy)-[1,2,4]oxadiazol-5-yl]-piperidine hydrochloride (80 mg, 0.27 mmol) in dry dichloromethane (5 mL), triethylamine (93 μL, 0.7 mmol) and 4-fluorobenzoyl chloride (31.4 μL, 0.27 mmol) were added dropwise at 0° C. The reaction mixture was allowed to warm at room temperature and stirred for 1.5 h under nitrogen atmosphere. The solvent was evaporated to dryness and the crude was purified by flash chromatography (silica gel, eluent: petroleum ether/AcOEt 70:30) to give 92 mg of the title compound,

LCMS (RT): 5.6 min (Method E); MS (ES+) gave m/z: 386.0 (MH+).

Compounds in Table 3 were prepared following the procedures described in Example 22 (C), starting from (S)-3-(3-fluorophenoxy-[1,2,4]oxadiazol-5-yl)-piperidine hydrochloride, prepared as described in Example 43 (B), and the corresponding commercially available carboxylic acids.

TABLE 3

| Ex. | Name | Analytical details |
|---|---|---|
| 44 | {(S)-3-[3-(3-Fluoro- | LCMS (RT): 5.84 min (Method E); MS (ES+) |
| | phenoxy)-[1,2,4]oxadiazol- | gave m/z: 381.9 (MH+) |
| | 5-yl]-piperidin-1-yl}-p- | |
| | tolyl-methanone | |
| 45 | {(S)-3-[3-(3-Fluoro- | LCMS (RT): 5.52 min (Method E); MS (ES+) |
| | phenoxy)-[1,2,4]oxadiazol- | gave m/z: 397.9 (MH+) |
| | 5-yl]-piperidin-1-yl}-(2- | |
| | methoxy-phenyl)-methanone | |
| 46 | {(S)-3-[3-(3-Fluoro- | LCMS (RT): 5.2 min (Method E); MS (ES+) |
| | phenoxy)-[1,2,4]oxadiazol- | gave m/z: 387 (MH+) |
| | 5-yl]-piperidin-1-yl}-(2- | |
| | fluoro-pyridin-4-yl)-methanone | |
| 47 | {(S)-3-[3-(3-Fluoro- | LCMS (RT): 3.5 min (Method E); MS (ES+) |
| | phenoxy)-[1,2,4]oxadiazol- | gave m/z: 358.0 (MH+) |
| | 5-yl]-piperidin-1-yl}-(3H- | |
| | imidazol-4-yl)-methanone | |
| 48 | (3,5-Difluoro-phenyl)-{(S)- | LCMS (RT): 5.8 min (Method E); MS (ES+) |
| | 3-[3-(3-fluoro-phenoxy)- | gave m/z: 403.9 (MH+) |
| | [1,2,4]oxadiazol-5-yl]- | |
| | piperidin-1-yl}-methanone | |
| 49 | {(S)-3-[3-(3-Fluoro- | LCMS (RT): 5.05 min (Method E); MS (ES+) |
| | phenoxy)-[1,2,4]oxadiazol- | gave m/z: 372.9 (MH+) |
| | 5-yl]-piperidin-1-yl}-(5- | |
| | methyl-isoxazol-4-yl)-methanone | |
| 50 | {(S)-3-[3-(3-Fluoro- | LCMS (RT): 4.76 min (Method E); MS (ES+) |
| | phenoxy)-[1,2,4]oxadiazol- | gave m/z: 374.9 (MH+) |
| | 5-yl]-piperidin-1-yl}- | |
| | thiazol-5-yl-methanone | |
| 51 | {(S)-3-[3-(3-Fluoro- | LCMS (RT): 5.1 min (Method E); MS (ES+) |
| | phenoxy)-[1,2,4]oxadiazol- | gave m/z: 387.0 (MH+) |
| | 5-yl]-piperidin-1-yl}-(6- | |
| | fluoro-pyridin-3-yl)-methanone | |
| 52 | {(S)-3-[3-(3-Fluoro- | LCMS (RT): 4.7 min (Method E); MS (ES+) |
| | phenoxy)-[1,2,4]oxadiazol- | gave m/z: 369.0 (MH+) |
| | 5-yl]-piperidin-1-yl}- | |
| | pyridin-2-yl-methanone | |

Compounds in Table 4 were prepared following the procedures described in Example 43 (C), starting from (S)-3-(3-fluorophenoxy-[1,2,4]oxadiazol-5-yl)-piperidine hydrochloride, prepared as described in Example 43 (B), and the corresponding commercially available acyl chlorides.

TABLE 4

| Ex. | Name | Analytical details |
|---|---|---|
| 53 | {(S)-3-[3-(3-Fluoro- | LCMS (RT): 5.5 min (Method E); MS (ES+) |
| | phenoxy)-[1,2,4]oxadiazol- | gave m/z: 367.9 (MH+) |
| | 5-yl]-piperidin-1-yl}- | |
| | phenyl-methanone | |
| 54 | (4-Chloro-phenyl)-{(S)-3- | LCMS (RT): 5.98 min (Method E); MS (ES+) |
| | [3-(3-fluoro-phenoxy)- | gave m/z: 401.9 (MH+) |
| | [1,2,4]oxadiazol-5-yl]- | |
| | piperidin-1-yl}-methanone | |
| 55 | {(S)-3-[3-(3-Fluoro- | LCMS (RT): 5.55 min (Method E); MS (ES+) |
| | phenoxy)-[1,2,4]oxadiazol- | gave m/z: 397.93 (MH+) |
| | 5-yl]-piperidin-1-yl}-(4- | |
| | methoxy-phenyl)-methanone | |
| 56 | (3,4-Dichloro-phenyl)-{(S)- | LCMS (RT): 6.34 min (Method E); MS (ES+) |
| | 3-[3-(3-fluoro-phenoxy)- | gave m/z: 435.84 and 437.82 (MH+) |
| | [1,2,4]oxadiazol-5-yl]- | |
| | piperidin-1-yl}-methanone | |
| 57 | {(S)-3-[3-(3-Fluoro- | LCMS (RT): 5.61 min (Method E); MS (ES+) |
| | phenoxy)-[1,2,4]oxadiazol- | gave m/z: 397.93 (MH+) |
| | 5-yl]-piperidin-1-yl}-(3- | |
| | methoxy-phenyl)-methanone | |
| 58 | {(S)-3-[3-(3-Fluoro- | LCMS (RT): 5.77 min (Method E); MS (ES+) |
| | phenoxy)-[1,2,4]oxadiazol- | gave m/z: 381.9 (MH+) |
| | 5-yl]-piperidin-1-yl}-o- | |
| | tolyl-methanone | |
| 59 | {(S)-3-[3-(3-Fluoro- | LCMS (RT): 5.61 min (Method E); MS (ES+) |
| | phenoxy)-[1,2,4]oxadiazol- | gave m/z: 385.9 (MH+) |
| | 5-yl]-piperidin-1-yl}-(2- | |
| | fluoro-phenyl)-methanone | |
| 60 | {(S)-3-[3-(3-Fluoro- | LCMS (RT): 5.67 min (Method E); MS (ES+) |
| | phenoxy)-[1,2,4]oxadiazol- | gave m/z: 385.9 (MH+) |
| | 5-yl]-piperidin-1-yl}-(3- | |
| | fluoro-phenyl)-methanone | |
| 61 | {(S)-3-[3-(3-Fluoro- | LCMS (RT): 3.9 min (Method E); MS (ES+) |
| | phenoxy)-[1,2,4]oxadiazol- | gave m/z: 369 (MH+) |
| | 5-yl]-piperidin-1-yl}- | |
| | pyridin-3-yl-methanone | |
| 62 | {(S)-3-[3-(3-Fluoro- | LCMS (RT): 3.7 min (Method E); MS (ES+) |
| | phenoxy)-[1,2,4]oxadiazol- | gave m/z: 369 (MH+) |
| | 5-yl]-piperidin-1-yl}- | |
| | pyridin-4-yl-methanone | |
| 63 | {(S)-3-[3-(3-Fluoro- | LCMS (RT): 5.1 min (Method E); MS (ES+) |
| | phenoxy)-[1,2,4]oxadiazol- | gave m/z: 387.0 (MH+) |
| | 5-yl]-piperidin-1-yl}-(3,5- | |
| | dimethyl-isoxazol-4-yl)-methanone | |

Pharmacology:

The compounds provided in the present invention are positive allosteric modulators of mGluR5. As such, these compounds do not activate the mGluR5 by themselves. Instead, the response of mGluR5 to a concentration of glutamate or mGluR5 agonist is increased when compounds of formula I are present. Compounds of formula I are expected to have their effect at mGluR5 by virtue of their ability to enhance the function of the receptor.

### Example A

### mGlaR65 Assay on Rat Cultured Cortical Astrocytes

Under exposure to growth factors (basic fibroblast growth factor, epidermal growth factor), rat cultured astrocytes express group I-Gq coupled mGluR transcripts, namely mGluR5, but none of the splice variants of mGluR1, and as a consequence, a functional expression of mGluR5 receptors (Miller et al. (1995) J. Neurosci. 15:6103-9): The stimulation of mGluR5 receptors with selective agonist CHPG and the full blockade of the glutamate-induced phosphoinositide (PI) hydrolysis and subsequent intracellular calcium mobilization with specific antagonist as MPEP confirm the unique expression of mGluR5 receptors in this preparation.

This preparation was established and used in order to assess the properties of the compounds of the present invention to increase the Ca²⁺ mobilization-induced by glutamate without showing any significant activity when applied in the absence of glutamate.

### Primary Cortical Astrocytes Culture:

Primary glial cultures were prepared from cortices of Sprague-Dawley 16 to 19 days old embryos using a modification of methods described by Mc Carthy and de Vellis (1980) J. Cell Biol. 85:890-902 and Miller et al. (1995) J. Neurosci. 15 (9):6103-9. The cortices were dissected and then dissociated by trituration in a sterile buffer containing 5.36 mM KCl, 0.44 mM NaHCO₃, 4.17 mM KH₂PO₄, 137 mM NaCl, 0.34 mM NaH₂PO₄, 1 g/L glucose. The resulting cell homogenate was plated onto poly-D-lysine precoated T175 flasks (BIOCOAT, Becton Dickinson Biosciences, Erembodegem, Belgium) in Dubelcco's Modified Eagle's Medium (D-MEM GlutaMAX™ I, Invitrogen, Basel, Switzerland) buffered with 25 mM HEPES and 22.7 mM NaHCO₃, and supplemented with 4.5 g/L glucose, 1 mM pyruvate and 15% fetal bovine serum (FBS, Invitrogen, Basel, Switzerland), penicillin and streptomycin and incubated at 37° C. with 5% CO₂. For subsequent seeding, the FBS supplementation was reduced to 10%. After 12 days, cells were subplated by trypsinisation onto poly-D-lysine precoated 384-well plates at a density of 20.000 cells per well in culture buffer.

### Ca²⁺ Mobilization Assay Using Rat Cortical Astrocytes:

After one day of incubation, cells were washed with assay buffer containing: 142 mM NaCl, 6 mM KCl, 1 mM Mg₂SO₄, 1 mM CaCl₂, 20 mM HEPES, 1 g/L glucose, 0.125 mM sulfinpyrazone, pH 7.4. After 60 min of loading with 4 μM Fluo-4 (Teffabs, Austin, Tex.), the cells were washed three times with 50 μl of PBS Buffer and resuspended in 45 μl of assay Buffer. The plates were then transferred to a Fluorometric Imaging Plate Reader (FLIPR, Molecular Devices, Sunnyvale, Calif.) for the assessment of intracellular calcium flux. After monitoring the baseline fluorescence for 10 s, a solution containing 10 μM of representative compound of the present invention diluted in Assay Buffer (15 μl of 4× dilutions) was added to the cell plate in the absence or in the presence of 300 nM of glutamate. Under these experimental conditions, this concentration induces less than 20% of the maximal response of glutamate and was the concentration used to detect the positive allosteric modulator properties of the compounds from the present invention. The final DMSO concentration in the assay was 0.3%. In each experiment, fluorescence was then monitored as a function of time for 3 minutes and the data analyzed using Microsoft Excel and GraphPad Prism. Each data point was also measured two times.

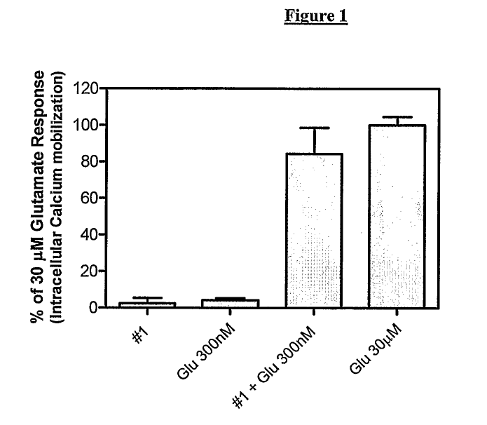

The results in FIG. 1 represent the effect of 10 μM of Example # 1 on primary cortical mGluR5-expressing cell cultures in the absence or in the presence of 300 nM glutamate. Data are expressed as the percentage of maximal response observed with 30 μM glutamate applied to the cells. Each bar graph is the mean and S.E.M of duplicate data points and is representative of three independent experiments

The results shown in Example A demonstrate that the compounds described in the present invention do not have an effect per se on mGluR5. Instead, when compounds are added together with an mGluR5 agonist such as glutamate, the effect measured is significantly potentiated compared to the effect of the agonist alone at the same concentration. This data indicates that the compounds of the present invention are positive allosteric modulators of mGluR5 receptors in native preparations.

### Example B

### mGluR5 Assay on HEK-Expressing Rat mGluR5

### Cell Culture

Positive functional expression of HEK-293 cells stably expressing rat mGluR5 receptor was determined by measuring intracellular Ca²⁺ changes using a Fluorometric Imaging Plate Reader (FLIPR, Molecular Devices, Sunnyvale, Calif.) in response to glutamate or selective known mGluR5 agonists and antagonists. Rat mGluR5RT-PCR products in HEK-293 cells were sequenced and found 100% identical to rat mGluR5Genbank reference sequence (NM_{—}017012). HEK-293 cells expressing rmGluR5 were maintained in media containing DMEM, dialyzed Fetal Bovine Serum (10%), Glutamax™ (2 mM), Penicillin (100 units/ml), Streptomycin (100 μg/ml), Geneticin (100 μg/ml) and Hygromycin-B (40 μg/ml) at 37° C./5% CO2.

### Fluorescent Cell Based-Ca²⁺ Mobilization Assay

After one day of incubation, cells were washed with assay buffer containing: 142 mM NaCl, 6 mM KCl, 1 mM Mg₂SO₄, 1 mM CaCl₂, 20 mM HEPES, 1 g/L glucose, 0.125 mM sulfinpyrazone, pH 7.4. After 60 min of loading with 4 uM Fluo-4 (TefLabs, Austin, Tex.), the cells were washed three times with 50 μl of PBS Buffer and resuspended in 45 μl of assay Buffer. The plates were then transferred to a Fluorometric Imaging Plate Reader (FLIPR, Molecular Devices, Sunnyvale, Calif.) for the assessment of intracellular calcium flux. After monitoring the baseline fluorescence for 10 seconds, increasing concentrations of representative compound (from 0.01 to 60 μM) of the present invention diluted in Assay Buffer (15 μl of 4× dilutions) was added to the cell. The final DMSO concentration in the assay was 0.3%. In each experiment, fluorescence was then monitored as a function of time for 3 minutes and the data analyzed using Microsoft Excel and GraphPad Prism. Each data point was also measured two times.

Under these experimental conditions, this HEK-rat mGluR5 cell line is able to directly detect positive allosteric modulators without the need of co-addition of glutamate or mGluR5 agonist. Thus, DFB, CPPHA and CDPPB, published reference positive allosteric modulators that are inactive in rat cortical astrocytes culture in the absence of added glutamate (Liu et al (2006) Eur. J. Pharmacol. 536:262-268; Zhang et al (2005); J. Pharmacol. Exp. Ther. 315:1212-1219) are activating, in this system, rat mGluR5 receptors.

The concentration-response curves of representative compounds of the present invention were generated using the Prism GraphPad software (Graph Pad Inc, San Diego, USA). The curves were fitted to a four-parameter logistic equation:

(Y=Bottom+(Top−Bottom)/(1+10̂((Log EC₅₀−X)*Hill Slope)

allowing determination of EC₅₀ values.

The Table 5 below represents the mean EC₅₀ obtained from at least three independent experiments of selected molecules performed in duplicate.

TABLE 5

| EXAMPLE | Ca++ Flux* |
|---|---|
| 1 | +++ |
| 2 | +++ |
| 3 | +++ |
| 4 | +++ |
| 5 | ++ |
| 6 | + |
| 7 | +++ |
| 8 | + |
| 9 | ++ |
| 10 | ++ |
| 11 | ++ |
| 12 | ++ |
| 13 | ++ |
| 14 | +++ |
| 15 | ++ |
| 16 | +++ |
| 17 | +++ |
| 18 | ++ |
| 19 | +++ |
| 20 | + |
| 21 | +++ |
| 22 | ++ |

*Table legend:
(+): EC₅₀ > 10 μM
(++): 1 μM < EC₅₀ < 10 μM
(+++): EC₅₀ < 1 μM

### Example C

### mGluR5 Binding Assay

Activity of compounds of the invention was examined following a radioligand binding technique using whole rat brain and tritiated 2-methyl-6-(phenylethynyl)-pyridine ([³H]-MPEP) as a ligand following similar methods than those described in Gasparini et al. (2002) Bioorg. Med. Chem. Lett. 12:407-409 and in Anderson et al. (2002) J. Pharmacol. Exp. Ther. 303 (3) 1044-1051.

### Membrane Preparation:

Cortices were dissected out from brains of 200-300 g Sprague-Dawley rats (Charles River Laboratories, L'Arbresle, France). Tissues were homogenized in 10 volumes (vol/wt) of ice-cold 50 mM HEPES-NaOH (pH 7.4) using a Polytron disrupter (Kinematica AG, Luzern, Switzerland) and centrifuged for 30 min at 40,000 g. (4° C.). The supernatant was discarded and the pellet washed twice by resuspension in 10 volumes 50 mM HEPES-NaOH. Membranes were then collected by centrifugation and washed before final resuspension in 10 volumes of 20 mM HEPES-NaOH, pH 7.4. Protein concentration was determined by the Bradford method (Bio-Rad protein assay, Reinach, Switzerland) with bovine serum albumin as standard.

[³H]-MPEP binding Experiments:

Membranes were thawed and resuspended in binding buffer containing 20 mM HEPES-NaOH, 3 mM MgCl₂, 3 mM CaCl₂, 100 mM NaCl, pH 7.4. Competition studies were carried out by incubating for 1 h at 4° C.: 3 nM [³H]-MPEP (39 Ci/mmol, Tocris, Cookson Ltd, Bristol, U.K.), 50 μg membrane and a concentration range of 0.003 nM-30 μM of compounds, for a total reaction volume of 300 μl. The non-specific binding was defined using 30 μM MPEP. Reaction was terminated by rapid filtration over glass-fiber filter plates (Unifilter 96-well GF/B filter plates, Perkin-Elmer, Schwerzenbach, Switzerland) using 4×400 μl ice cold buffer using cell harvester (Filtermate, Perkin-Elmer, Downers Grove, USA). Radioactivity was determined by liquid scintillation spectrometry using a 96-well plate reader (TopCount, Perkin-Elmer, Downers Grove, USA).

### Data Analysis:

The inhibition curves were generated using the Prism GraphPad program (Graph Pad Software Inc, San Diego, USA). IC₅₀ determinations were made from data obtained from 8 point-concentration response curves using a non linear regression analysis. The mean of IC₅₀ obtained from at least three independent experiments of selected molecules performed in duplicate were calculated.

The compounds of this application have IC₅₀ values in the range of less than 100 μM. Example # 1 has IC₅₀ value of less than 30 μM.

The results shown in Examples A, B and C demonstrate that the compounds described in the present invention are positive allosteric modulators of rat mGluR5 receptors. These compounds are active in native systems and are able to inhibit the binding of the prototype mGluR5 allosteric modulator [³H]-MPEP known to bind remotely from the glutamate binding site into the transmembrane domains of mGluR5 receptors (Malherbe et al (2003) Mol. Pharmacol. 64(4):823-32)

Thus, the positive allosteric modulators provided in the present invention are expected to increase the effectiveness of glutamate or mGluR5 agonists at mGluR5 receptor. Therefore, these positive allosteric modulators are expected to be useful for treatment of various neurological and psychiatric disorders associated with glutamate dysfunction described to be treated herein and others that can be treated by such positive allosteric modulators.

The compounds of the present invention are positive allosteric modulators of mGluR5 receptors, they are useful for the production of medications, especially for the prevention or treatment of central nervous system disorders as well as other disorders modulated by this receptor.

The compounds of the invention can be administered either alone, or in combination with other pharmaceutical agents effective in the treatment of conditions mentioned above.

### Formulation Examples

Typical examples of recipes for the formulation of the invention are as follows:

1) Tablets

| | | |
|---|---|---|
| Compound of the example 1 | 5 to 50 | mg |
| Di-calcium phosphate | 20 | mg |
| Lactose | 30 | mg |
| Talcum | 10 | mg |
| Magnesium stearate | 5 | mg |
| Potato starch | ad 200 | mg |

In this example, the compound of the example 1 can be replaced by the same amount of any of the described examples 1 to 63.

2) Suspension:

An aqueous suspension is prepared for oral administration so that each 1 milliliter contains 1 to 5 mg of one of the described example, 50 mg of sodium carboxymethyl cellulose, 1 mg of sodium benzoate, 500 mg of sorbitol and water ad 1 ml.

3) Injectable

A parenteral composition is prepared by stirring 1.5% by weight of active ingredient of the invention in 10% by volume propylene glycol and water.

4) Ointment

| | | |
|---|---|---|
| Compound of the example 1 | 5 to 1000 | mg |
| Stearyl alcohol | 3 | g |
| Lanoline | 5 | g |
| White petroleum | 15 | g |
| Water | ad 100 | g |

In this example, the compound of the example 1 can be replaced by the same amount of any of the described examples 1 to 63.

Reasonable variations are not to be regarded as a departure from the scope of the invention. It will be obvious that the thus described invention may be varied in many ways by those skilled in the art.

1. A compound of general formula I: 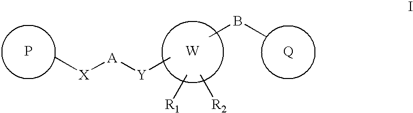
Wherein
W represents (C₅-C₇)cycloalkyl, (C₅-C₇)heterocycloalkyl or (C₅-C₇)heterocycloalkenyl ring;
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P and Q are each independently selected and denote a cycloalkyl, a heterocycloalkyl, an aryl or heteroaryl group of formula 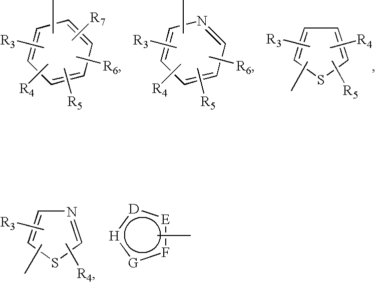
R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
A is azo —N═N—, ethyl, ethenyl, ethynyl, —NR₈C(═O)—, —NR₈S(═O)₂—, —C(═O)NR₈—, —S—, —S(═O)—, —S(═O)₂—, —S(═O)₂NR₈—, —C(═O)—O—, —O—C(═O)—, —C(═NR₈)NR₉—, —C(═NOR₈)NR₉—, —NR₈C(═NOR₉)—, ═N—O—, —O—N═CH— or a group aryl or heteroaryl of formula 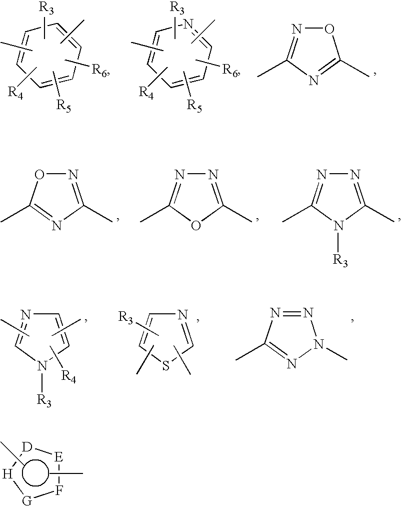
R₃, R₄, R₅ and R₆ independently are as defined above;
D, E, F, G and H in A independently represent a carbon group, oxygen, nitrogen, sulphur or a double bond;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
X and Y are each independently selected from a bond, —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR₁₁C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁, —(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
X and Y together cannot be a bond;
R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
Any N may be an N-oxide;
or pharmaceutically acceptable salts, hydrates or solvates of such compounds.

2. A compound according to claim 1 having the formula I-A 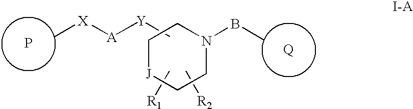
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P and Q are each independently selected and denote a cycloalkyl, a heterocycloalkyl, an aryl or heteroaryl group of formula 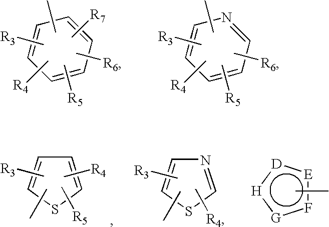
R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—CO—C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
A is azo —N═N—, ethyl, ethenyl, ethynyl, —NR₈C(═O)—, —NR₈S(═O)₂—, —C(═O)NR₈—, —S—, —S(═O)—, —S(═O)₂—, —S(═O)₂NR₈—, —C(═O)—O—, —O—C(═O)—, —C(═NR₈)NR₉—, —C(═NOR₈)NR₉—, —NR₈C(═NOR₉)—, ═N—O—, —O—N═CH— or a group aryl or heteroaryl of formula 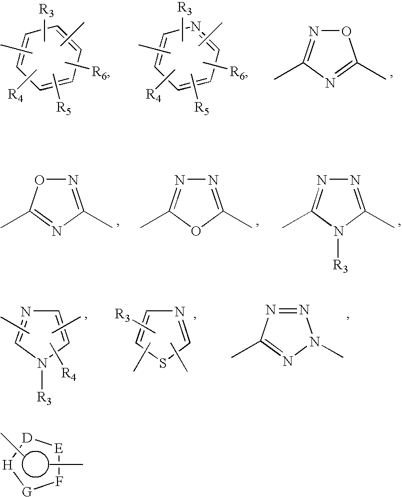
R₃, R₄, R₅ and R₆ independently are as defined above;
D, E, F, G and H in A independently represent a carbon group, oxygen, nitrogen, sulphur or a double bond;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
X and Y are each independently selected from a bond, —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₅)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR₁₁C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, (C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
X and Y together cannot be a bond;
R₁₁ and R₁₂ each independently is hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆-alkyl), —O—(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

3. A compound according to claim 1 having the formula I-B 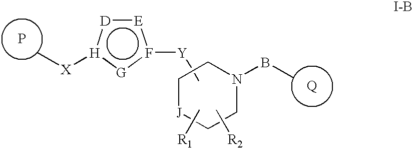
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P and Q are each independently selected and denote a cycloalkyl, a heterocycloalkyl, an aryl or heteroaryl group of formula 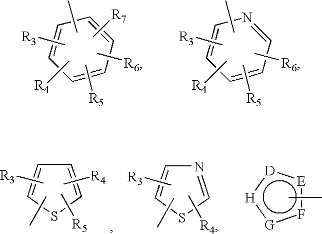
R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
X and Y are each independently selected from a bond, —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR₁₁C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁, C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
X and Y together cannot be a bond;
R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

4. A compound according to claim 1 having the formula I-C 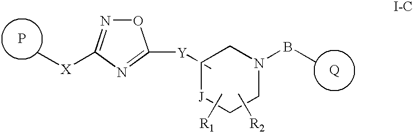
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P and Q are each independently selected and denote a cycloalkyl, a heterocycloalkyl, an aryl or heteroaryl group of formula 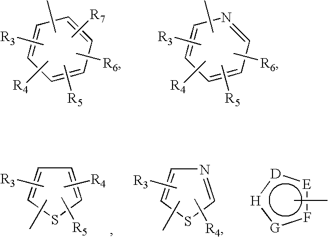
R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
X and Y are each independently selected from a bond, —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR₁₁C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl
X and Y together cannot be a bond;
R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

5. A compound according to claim 1 shaving the formula I-D 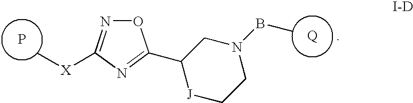
Wherein
P and Q are each independently selected and denote a cycloalkyl, a heterocycloalkyl, an aryl or heteroaryl group of formula 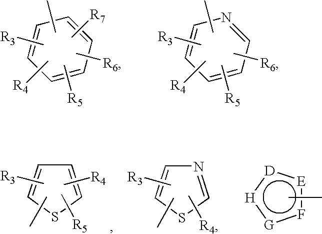
R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
X represents —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, (C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR¹¹C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁, —(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

6. A compound according to claim 5 having the formula I-D
Wherein
X represents an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl- or (C₀-C₆)alkyl-NR₁₁—(C₄-C₁₀)alkylcycloalkyl-;
R₁₁ is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆ alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
Any N may be an N-oxide;
or pharmaceutically acceptable salts, hydrates or solvates of such compounds.

7. A compound according to claim 1 having the formula II-A 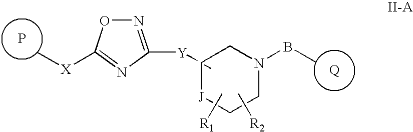
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P and Q are each independently selected and denote a cycloalkyl, a heterocycloalkyl, an aryl or heteroaryl group of formula 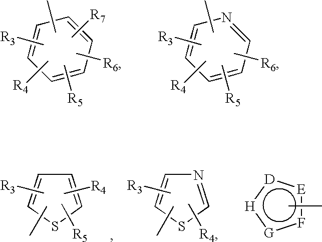
R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
X and Y are each independently selected from a bond, —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, (C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁, —(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁—(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR₁₁C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁₁—(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₁-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁₁C(═O)O—(C₃-C₇)cycloalkyl- or —(C₀-C₆)alkyl-NR₁₁C(═O)O—(C₄-C₁₀)alkylcycloalkyl;
X and Y together cannot be a bond;
R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

8. A compound according to claim 1 having the formula II-B 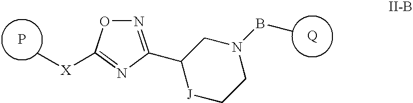
Wherein
P and Q are each independently selected and denote a cycloalkyl, a heterocycloalkyl, an aryl or heteroaryl group of formula 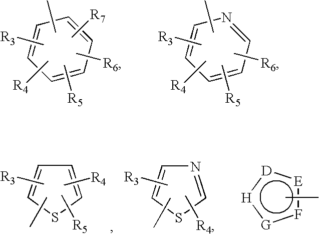
R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
X represents —NR₁₁C(═O)O—, an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-C(═O)O—(C₀-C₆)alkyl-, —(C₀-C₆)alkyl-C(═O)O—(C₂-C₆)alkynyl-, —(C₀-C₆)alkyl-C(═O)O—(C₂-C₆)alkenyl-, —(C₀-C₆)alkyl-C(═O)O—(C₃-C₇)cycloalkyl-, —(C₀-C₆)alkyl-C(═O)O—(C₄-C₁₀)alkylcycloalkyl-, —(C₀-C₆)alkyl-C(═O)NR₁₁—(C₀-C₆)alkyl-, —(C₀-C₆)alkyl-C(═O)NR₁₁—(C₂-C₆)alkynyl-, —(C₀-C₆)alkyl-C(═O)NR₁₁—(C₂-C₆)alkenyl-, —(C₀-C₆)alkyl-C(═O)NR₁₁—(C₃-C₇)cycloalkyl-, —(C₀-C₆)alkyl-C(═O)NR₁, —(C₄-C₁₀)alkylcycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-S(O)₂NR₁₁—(C₀-C₆)alkyl-, —(C₀-C₆)alkyl-S(O)₂NR₁₁—(C₂-C₆)alkynyl-, —(C₀-C₆)alkyl-S(O)₂NR₁₁—(C₂-C₆)alkenyl-, —(C₀-C₆)alkyl-S(O)₂NR₁₁—(C₃-C₇)cycloalkyl-, —(C₀-C₆)alkyl-S(O)₂NR₁₁—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl-, —(C₀-C₆)alkyl-NR₁₁—(C₄-C₁₀)alkylcycloalkyl-, —(C₀-C₆)alkyl-NR₁₁C(═O)—(C₀-C₆)alkyl-, —(C₀-C₆)alkyl-NR¹¹C(═O)—(C₂-C₆)alkynyl-, —(C₀-C₆)alkyl-NR₁₁C(═O)—(C₂-C₆)alkenyl-, —(C₀-C₆)alkyl-NR₁₁C(═O)—(C₃-C₇)cycloalkyl-, —(C₀-C₆)alkyl-NR₁₁C(═O)—(C₄-C₁₀)alkylcycloalkyl-, —(C₀-C₆)alkyl-NR₁₂C(═O)NR₁₁—(C₀-C₆)alkyl-, —(C₀-C₆)alkyl-NR₁₂C(═O)NR₁₁—(C₂-C₆)alkynyl-, —(C₀-C₆)alkyl-NR₁₂C(═O)NR₁₁—(C₂-C₆)alkenyl-, —(C₀-C₆)alkyl-NR₁₂C(═O)NR₁₁—(C₃-C₇)cycloalkyl-, —(C₀-C₆)alkyl-NR₁₂C(═O)NR₁₁—(C₄-C₁₀)alkylcycloalkyl-, —(C₀-C₆)alkyl-NR₁₁S(O)₂—(C₀-C₆)alkyl-, —(C₀-C₆)alkyl-NR₁₁S(O)₂—(C₂-C₆)alkynyl-, —(C₀-C₆)alkyl-NR₁₁S(O)₂—(C₂-C₆)alkenyl-, —(C₀-C₆)alkyl-NR₁₁S(O)₂—(C₃-C₇)cycloalkyl-, —(C₀-C₆)alkyl-NR₁₁S(O)₂—(C₄-C₁₀)alkylcycloalkyl-, —(C₀-C₆)alkyl-NR₁₂C(═S)NR₁, —(C₀-C₆)alkyl-, —(C₀-C₆)alkyl-NR₁₂C(═S)NR₁₁—(C₂-C₆)alkynyl-, —(C₀-C₆)alkyl-NR₁₂C(═S)NR₁₁—(C₂-C₆)alkenyl-, —(C₀-C₆)alkyl-NR₁₂C(═S)NR₁₁—(C₃-C₇)cycloalkyl-, —(C₀-C₆)alkyl-NR₁₂C(═S)NR₁₁—(C₄-C₁₀)alkylcycloalkyl-, —(C₀-C₆)alkyl-OC(═O)—(C₀-C₆)alkyl-, —(C₀-C₆)alkyl-OC(═O)—(C₂-C₆)alkynyl-, —(C₀-C₆)alkyl-OC(═O)—(C₂-C₆)alkenyl-, —(C₀-C₆)alkyl-OC(═O)—(C₄-C₁₀)alkylcycloalkyl-, —(C₀-C₆)alkyl-OC(═O)—(C₃-C₇)cycloalkyl-, —(C₀-C₆)alkyl-OC(═O)NR₁₁—(C₀-C₆)alkyl-, —(C₀-C₆)alkyl-OC(═O)NR₁₁—(C₂-C₆)alkynyl-, —(C₀-C₆)alkyl-OC(═O)NR₁—(C₂-C₆)alkenyl-, —(C₀-C₆)alkyl-OC(═O)NR₁₁—(C₄-C₁₀)alkylcycloalkyl-, —(C₀-C₆)alkyl-OC(═O)NR₁₁—(C₃-C₇)cycloalkyl-, —(C₀-C₆)alkyl-NR₁₁C(═O)O—(C₀-C₆)alkyl-, —(C₀-C₆)alkyl-NR₁₁C(═O)O—(C₂-C₆)alkynyl-, —(C₀-C₆)alkyl-NR₁₁C(═O)O—(C₂-C₆)alkenyl-, —(C₀-C₆)alkyl-NR₁, C(═O)O—(C₃-C₇)cycloalkyl- or —(C₀-C₆)alkyl-NR₁₁C(═O)O—(C₄-C₁₀)alkylcycloalkyl;
R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

9. A compound according to claim 1 shaving the formula II-B
Wherein
X represents an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-NR₁₁—(C₀-C₆)alkyl-, —(C₀-C₆)alkyl-NR₁—(C₂-C₆)alkynyl-, —(C₀-C₆)alkyl-NR₁₁—(C₂-C₆)alkenyl-, —(C₀-C₆)alkyl-NR₁, —(C₃-C₇)cycloalkyl- or —(C₀-C₆)alkyl-NR₁₁—(C₄-C₁₀)alkylcycloalkyl-;
R₁₁ is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
Any N may be an N-oxide;
or pharmaceutically acceptable salts, hydrates or solvates of such compounds.

10. A compound according to claim 1 shaving the formula III-A 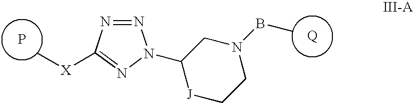
Wherein
P and Q are each independently selected and denote a cycloalkyl, a heterocycloalkyl, an aryl or heteroaryl group of formula 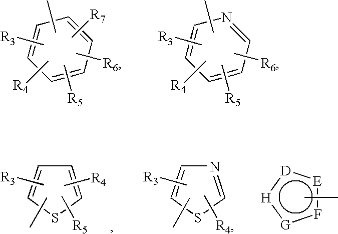
R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
X represents an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl- or —(C₀-C₆)alkyl-NR₁₁—(C₄-C₁₀)alkylcycloalkyl-;
R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

11. A compound according to claim 1 having the formula IV-A 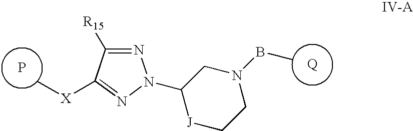
Wherein
P and Q are each independently selected and denote a cycloalkyl, a heterocycloalkyl, an aryl or heteroaryl group of formula 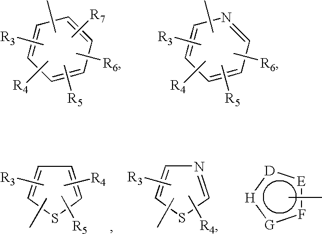
R₃, R₄, R₅, R₆, and R₇ independently are hydrogen, halogen, —CN, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, N(═NR₁₀)NR₈R₉, —NR₈COR₉, NR₈CO₂R₉, NR₈SO₂R₉, —NR₁₀CONR₈R₉, —SR₈, —S(═O)R₈, —S(═O)₂R₈, —S(═O)₂NR₈R₉, —C(═O)R₈, —COOR₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G and H in P and Q represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉—S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
X represents an optionally substituted —(C₁-C₆)alkyl-, —(C₂-C₆)alkynyl-, —(C₂-C₆)alkenyl-, —(C₃-C₇)cycloalkyl-, —(C₃-C₈)cycloalkenyl-, —(C₁-C₆)alkylhalo-, —(C₁-C₆)alkylcyano-, —(C₀-C₆)alkyl-O—(C₀-C₆)alkyl-, —(C₀-C₆)alkyl-O—(C₂-C₆)alkynyl-, —(C₀-C₆)alkyl-O—(C₂-C₆)alkenyl-, —(C₀-C₆)alkyl-O—(C₃-C₇)cycloalkyl-, —(C₀-C₆)alkyl-O—(C₄-C₁₀)alkylcycloalkyl-, —(C₀-C₆)alkyl-C(═O)—(C₀-C₆)alkyl-, —(C₀-C₆)alkyl-C(═O)—(C₂-C₆)alkynyl-, —(C₀-C₆)alkyl-C(═O)—(C₂-C₆)alkenyl-, —(C₀-C₆)alkyl-C(═O)—(C₃-C₇)alkylcycloalkyl-, —(C₀-C₆)alkyl-C(═O)—(C₄-C₁₀)cycloalkyl-, —(C₀-C₆)alkyl-S—(C₀-C₆)alkyl-, —(C₀-C₆)alkyl-S—(C₂-C₆)alkynyl-, —(C₀-C₆)alkyl-S—(C₂-C₆)alkenyl-, —(C₀-C₆)alkyl-S—(C₃-C₇)cycloalkyl-, —(C₀-C₆)alkyl-S—(C₄-C₁₀)alkylcycloalkyl-, —(C₀-C₆)alkyl-S(O)—(C₀-C₆)alkyl-, —(C₀-C₆)alkyl-O—(C₂-C₆)alkynyl-, —(C₀-C₆)alkyl-S(O)—(C₂-C₆)alkenyl-, —(C₀-C₆)alkyl-S(O)—(C₃-C₇)cycloalkyl-, —(C₀-C₆)alkyl-S(O)—(C₄-C₁₀)alkylcycloalkyl-, —(C₀-C₆)alkyl-S(O)₂—(C₀-C₆)alkyl-, —(C₀-C₆)alkyl-S(O)₂—(C₂-C₆)alkynyl-, —(C₀-C₆)alkyl-S(O)₂—(C₂-C₆)alkenyl-, —(C₀-C₆)alkyl-S(O)₂—(C₃-C₇)cycloalkyl-, —(C₀-C₆)alkyl-S(O)₂—(C₄-C₁₀)alkylcycloalkyl-, —(C₀-C₆)alkyl-NR₁₁—(C₀-C₆)alkyl-, —(C₀-C₆)alkyl-NR₁₁—(C₂-C₆)alkynyl-, —(C₀-C₆)alkyl-NR₁₁—(C₂-C₆)alkenyl-, —(C₀-C₆)alkyl-NR₁₁—(C₃-C₇)cycloalkyl- or —(C₀-C₆)alkyl-NR₁₁—(C₄-C₁₀)alkylcycloalkyl-;
R₁₁ and R₁₂ each independently is hydrogen, C₁-C₆-alkyl, C₃-C₆-cycloalkyl, C₃-C₇-cycloalkylalkyl, C₂-C₆-alkenyl, C₂-C₆-alkynyl, halo-C₁-C₆-alkyl, heterocycloalkyl, heteroaryl, heteroarylalkyl, arylalkyl or aryl; any of which is optionally substituted with 1-5 independent halogen, —CN, C₁-C₆-alkyl, —O(C₀-C₆-alkyl), —O(C₃-C₇-cycloalkylalkyl), —O(aryl), —O(heteroaryl), —N(C₀-C₆-alkyl)(C₀-C₆-alkyl), —N(C₀-C₆-alkyl)(C₃-C₇-cycloalkyl) or —N(C₀-C₆-alkyl)(aryl) substituents;
J represents a single bond, —C(R₁₃)(R₁₄), —O—, —N(R₁₃)— or —S—;
R₁₃, R₁₄ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₈)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
R₁₅ is hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl, —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

12. A compound according to claim 1, which can exist as optical isomers, wherein said compound is either the racemic mixture or an individual optical isomer.

13. A compound according to claim 1, wherein said compound is selected from:
{(S)-3-[3-(4-Fluoro-benzyl)-[1,2,4]oxadiazol-5-yl]-piperidin-1-yl}-(4-fluoro-phenyl)-methanone,
(3,4-Difluoro-phenyl)-{(S)-3-[3-(4-fluoro-benzyl)-[1,2,4]oxadiazol-5-yl]-piperidin-1-yl}-methanone
(3,4-Difluoro-phenyl)-{(S)-3-[5-(4-fluoro-benzyl)-[1,2,4]oxadiazol-3-yl]-piperidin-1-yl}-methanone
{(S)-3-[5-(4-Fluoro-benzyl)-[1,2,4]oxadiazol-3-yl]-piperidin-1-yl}-(4-fluoro-phenyl)-methanone
(4-Fluoro-phenyl)-{(S)-3-[5-((S)-1-phenyl-ethyl)-[1,2,4]oxadiazol-3-yl]-piperidin-1-yl}-methanone
(4-Fluoro-phenyl)-{(S)-3-[5-((R)-1-phenyl-ethyl)-[1,2,4]oxadiazol-3-yl]-piperidin-1-yl}-methanone
[(S)-3-(5-Benzyl-[1,2,4]oxadiazol-3-yl)-piperidin-1-yl]-(4-fluoro-phenyl)-methanone
(4-Fluoro-phenyl)-{(S)-3-[5-((S)-hydroxy-phenyl-methyl)-[1,2,4]oxadiazol-3-yl]-piperidin-1-yl}-methanone
(4-Fluoro-phenyl)-{(S)-3-[5-((R)-hydroxy-phenyl-methyl)-[1,2,4]oxadiazol-3-yl]-piperidin-1-yl}-methanone
(4-Fluoro-phenyl)-[(S)-3-(5-phenethyl-[1,2,4]oxadiazol-3-yl)-piperidin-1-yl]-methanone
{3-[(S)-1-(4-Fluoro-benzoyl)-piperidin-3-yl]-[1,2,4]oxadiazol-5-yl}-phenyl-methanone
(4-Fluoro-phenyl)-[(S)-3-(5-phenylamino-[1,2,4]oxadiazol-3-yl)-piperidin-1-yl]-methanone
{(S)-3-[5-(4-Fluoro-benzylamino)-[1,2,4]oxadiazol-3-yl]-piperidin-1-yl}-(4-fluoro-phenyl)-methanone
[(S)-3-(5-Benzyl-tetrazol-2-yl)-piperidin-1-yl]-(4-fluoro-phenyl)-methanone
{3-[3-(4-Fluoro-phenoxy)-[1,2,4]oxadiazol-5-yl]-piperidin-1-yl}-(4-fluoro-phenyl)-methanone
(4-Fluoro-phenyl)-[3-(3-phenoxy-[1,2,4]oxadiazol-5-yl)-piperidin-1-yl]-methanone
(6-Fluoro-pyridin-3-yl)-[(S)-3-(3-phenoxy-[1,2,4]oxadiazol-5-yl)-piperidin-1-yl]-methanone
{(S)-3-[3-(2-Fluoro-phenoxy)-[1,2,4]oxadiazol-5-yl]-piperidin-1-yl}-(6-fluoro-pyridin-3-yl)-methanone
{(S)-3-[3-(3-Fluoro-phenoxy)-[1,2,4]oxadiazol-5-yl]-piperidin-1-yl}-(6-fluoro-pyridin-3-yl)-methanone
(4-Fluoro-phenyl)-[(S)-3-(3-phenylsulfanyl-[1,2,4]oxadiazol-5-yl)-piperidin-1-yl]-methanone
{3-[3-(3-Fluoro-phenoxy)-[1,2,4]oxadiazol-5-yl]-piperidin-1-yl}-(4-fluoro-phenyl)-methanone
{3-[3-(3-Fluoro-phenoxy)-[1,2,4]oxadiazol-5-yl]-piperidin-1-yl}-(4-fluoro-phenyl)-methanone
(4-Methylphenyl)-[(S)-3-(3-phenoxy-[1,2,4]oxadiazol-5-yl)-piperidin-1-yl]-methanone
(2-Methoxy-phenyl)-[(S)-3-(3-phenoxy-[1,2,4]oxadiazol-5-yl)-piperidin-1-yl]-methanone
[(S)-3-(3-Phenoxy-[1,2,4]oxadiazol-5-yl)-piperidin-1-yl]-pyridin-2-yl-methanone
(2-Fluoro-pyridin-4-yl)-[(S)-3-(3-phenoxy-[1,2,4]oxadiazol-5-yl)-piperidin-1-yl]-methanone
(3H-Imidazol-4-yl-[(S)-3-(3-phenoxy-[1,2,4]oxadiazol-5-yl)-piperidin-1-yl]-methanone
(3,5-Difluoro-phenyl)-[(S)-3-(3-phenoxy-[1,2,4]oxadiazol-5-yl)-piperidin-1-yl]-methanone
(5-Methyl-isoxazol-4-yl)-[(S)-3-(3-phenoxy-[1,2,4]oxadiazol-5-yl)-piperidin-1-yl]-methanone
[(S)-3-(3-Phenoxy-[1,2,4]oxadiazol-5-yl)-piperidin-1-yl]-thiazol-5-yl-methanone
[(S)-3-(3-Phenoxy-[1,2,4]oxadiazol-5-yl)-piperidin-1-yl]-phenyl-methanone
(4-Chloro-phenyl)-[(S)-3-(3-phenoxy-[1,2,4]oxadiazol-5-yl)-piperidin-1-yl]-methanone
(4-Methoxy-phenyl)-[(S)-3-(3-phenoxy-[1,2,4]oxadiazol-5-yl)-piperidin-1-yl]-methanone
(3,4-Dichloro-phenyl)-[(S)-3-(3-phenoxy-[1,2,4]oxadiazol-5-yl)-piperidin-1-yl]-methanone
(3-Methoxy-phenyl)-[(S)-3-(3-phenoxy-[1,2,4]oxadiazol-5-yl)-piperidin-1-yl]-methanone
(2-Methyl-phenyl)-[(S)-3-(3-phenoxy-[1,2,4]oxadiazol-5-yl)-piperidin-1-yl]-methanone
(2-Fluoro-phenyl)-[(S)-3-(3-phenoxy-[1,2,4]oxadiazol-5-yl)-piperidin-1-yl]-methanone
(3-Fluoro-phenyl)-[(S)-3-(3-phenoxy-[1,2,4]oxadiazol-5-yl)-piperidin-1-yl]-methanone
[(S)-3-(3-Phenoxy-[1,2,4]oxadiazol-5-yl)-piperidin-1-yl]-pyridin-3-yl-methanone
[(S)-3-(3-Phenoxy-[1,2,4]oxadiazol-5-yl)-piperidin-1-yl]-pyridin-4-yl-methanone
(3,5-Dimethyl-isoxazol-4-yl)-[(S)-3-(3-phenoxy-[1,2,4]oxadiazol-5-yl)-piperidin-1-yl]-methanone
(4-Fluoro-phenyl)-[(S)-3-(3-phenoxy-[1,2,4]oxadiazol-5-yl)-piperidin-1-yl]-methanone {(S)-3-[3-(3-Fluoro-phenoxy)-[1,2,4]oxadiazol-5-yl]-piperidin-1-yl}-(4-fluoro-phenyl)-methanone
{(S)-3-[3-(3-Fluoro-phenoxy)-[1,2,4]oxadiazol-5-yl]-piperidin-1-yl}-p-tolyl-methanone
{(S)-3-[3-(3-Fluoro-phenoxy)-[1,2,4]oxadiazol-5-yl]-piperidin-1-yl}-(2-methoxy-phenyl)-methanone
{(S)-3-[3-(3-Fluoro-phenoxy)-[1,2,4]oxadiazol-5-yl]-piperidin-1-yl}-(2-fluoro-pyridin-4-yl)-methanone
{(S)-3-[3-(3-Fluoro-phenoxy)-[1,2,4]oxadiazol-5-yl]-piperidin-1-yl}-(3H-imidazol-4-yl)-methanone
(3,5-Difluoro-phenyl)-{(S)-3-[3-(3-fluoro-phenoxy)-[1,2,4]oxadiazol-5-yl]-piperidin-1-yl}-methanone
{(S)-3-[3-(3-Fluoro-phenoxy)-[1,2,4]oxadiazol-5-yl]-piperidin-1-yl}-(5-methyl-isoxazol-4-yl)-methanone
{(S)-3-[3-(3-Fluoro-phenoxy)-[1,2,4]oxadiazol-5-yl]-piperidin-1-yl}-thiazol-5-yl-methanone
{(S)-3-[3-(3-Fluoro-phenoxy)-[1,2,4]oxadiazol-5-yl]-piperidin-1-yl}-(6-fluoro-pyridin-3-yl)-methanone
{(S)-3-[3-(3-Fluoro-phenoxy)-[1,2,4]oxadiazol-5-yl]-piperidin-1-yl}-pyridin-2-yl-methanone
{(S)-3-[3-(3-Fluoro-phenoxy)-[1,2,4]oxadiazol-5-yl]-piperidin-1-yl}-phenyl-methanone
(4-Chloro-phenyl)-{(S)-3-[3-(3-fluoro-phenoxy)-[1,2,4]oxadiazol-5-yl]-piperidin-1-yl}-methanone
{(S)-3-[3-(3-Fluoro-phenoxy)-[1,2,4]oxadiazol-5-yl]-piperidin-1-yl}-(4-methoxy-phenyl)-methanone
(3,4-Dichloro-phenyl)-{(S)-3-[3-(3-fluoro-phenoxy)-[1,2,4]oxadiazol-5-yl]-piperidin-1-yl}-methanone
{(S)-3-[3-(3-Fluoro-phenoxy)-[1,2,4]oxadiazol-5-yl]-piperidin-1-yl}-(3-methoxy-phenyl)-methanone
{(S)-3-[3-(3-Fluoro-phenoxy)-[1,2,4]oxadiazol-5-yl]-piperidin-1-yl}-o-tolyl-methanone
{(S)-3-[3-(3-Fluoro-phenoxy)-[1,2,4]oxadiazol-5-yl]-piperidin-1-yl}-(2-fluoro-phenyl)-methanone
{(S)-3-[3-(3-Fluoro-phenoxy)-[1,2,4]oxadiazol-5-yl]-piperidin-1-yl}-(3-fluoro-phenyl)-methanone
{(S)-3-[3-(3-Fluoro-phenoxy)-[1,2,4]oxadiazol-5-yl]-piperidin-1-yl}-pyridin-3-yl-methanone
{(S)-3-[3-(3-Fluoro-phenoxy)-[1,2,4]oxadiazol-5-yl]-piperidin-1-yl}-pyridin-4-yl-methanone
{(S)-3-[3-(3-Fluoro-phenoxy)-[1,2,4]oxadiazol-5-yl]-piperidin-1-yl}-(3,5-dimethyl-isoxazol-4-yl)-methanone
and pharmaceutically acceptable salts thereof.

14. A pharmaceutical composition comprising a therapeutically effective amount of a compound according to claim 1 and a pharmaceutically acceptable carrier and/or excipient.

15. A method of treating or preventing a condition in a mammal, including a human, the treatment or prevention of which is affected or facilitated by the neuromodulatory effect of mGluR5 allosteric modulators, comprising administering to a mammal in need of such treatment or prevention, an effective amount of a compound according to claim 1.

16. A method of treating or preventing a condition in a mammal, including a human, the treatment or prevention of which is affected or facilitated by the neuromodulatory effect of mGluR5 positive allosteric modulators (enhancer), comprising administering to a mammal in need of such treatment or prevention, an effective amount of a compound according to claim 1.

17. A method useful for treating or preventing central nervous system disorders selected from the group consisting of anxiety disorders: Agoraphobia, Generalized Anxiety Disorder (GAD), Obsessive-Compulsive Disorder (OCD), Panic Disorder, Posttraumatic Stress Disorder (PTSD), Social Phobia, Other Phobias, Substance-Induced Anxiety Disorder, comprising administering an effective amount of a compound according to claim 1.

18. A method useful for treating or preventing central nervous system disorders selected from the group consisting of childhood disorders: Attention-Deficit/Hyperactivity Disorder), comprising administering an effective amount of a compound according to claim 1.

19. A method useful for treating or preventing central nervous system disorders selected from the group consisting of eating Disorders (Anorexia Nervosa, Bulimia Nervosa), comprising administering an effective amount of a compound according to claim 1.

20. A method useful for treating or preventing central nervous system disorders selected from the group consisting of mood disorders: Bipolar Disorders (I & II), Cyclothymic Disorder, Depression, Dysthymic Disorder, Major Depressive Disorder, Substance-Induced Mood Disorder, comprising administering an effective amount of a compound according to claim 1.

21. A method useful for treating or preventing central nervous system disorders selected from the group consisting of psychotic disorders: Schizophrenia, Delusional Disorder, Schizoaffective Disorder, Schizophreniform Disorder, Substance-Induced Psychotic Disorder, comprising administering an effective amount of a compound according to claim 1.

22. A method useful for treating or preventing central nervous system disorders selected from the group consisting of cognitive disorders: Delirium, Substance-induced Persisting Delirium, Dementia, Dementia Due to HIV Disease, Dementia Due to Huntington's Disease, Dementia Due to Parkinson's Disease, Dementia of the Alzheimer's Type, Substance-induced Persisting Dementia, Mild Cognitive Impairment, comprising administering an effective amount of a compound according to claim 1.

23. A method useful for treating or preventing central nervous system disorders selected from the group consisting of personality disorders: Obsessive-Compulsive Personality Disorder, Schizoid, Schizotypal disorder, comprising administering an effective amount of a compound according to claim 1.

24. A method useful for treating or preventing central nervous system disorders selected from the group consisting of substance-related disorders: Alcohol abuse, Alcohol dependence, Alcohol withdrawal, Alcohol withdrawal delirium, Alcohol-induced psychotic disorder, Amphetamine dependence, Amphetamine withdrawal, Cocaine dependence, Cocaine withdrawal, Nicotine dependence, Nicotine withdrawal, Opioid dependence, Opioid withdrawal, comprising administering an effective amount of a compound according to claim 1.

25. A method useful for treating or preventing inflammatory central nervous system disorders selected from multiple sclerosis form such as benign multiple sclerosis, relapsing-remitting multiple sclerosis, secondary progressive multiple sclerosis, primary progressive multiple sclerosis, progressive-relapsing multiple sclerosis, comprising administering an effective amount of a compound according to claim 1.

26-27. (canceled)
